(19) United States
(12) Patent Application Publication
(10) Pub. No.: US 2023/0312656 A1
(43) Pub. Date: Oct. 5, 2023

(54) RECOMBINANT SARS-COV-2 SPIKE PROTEIN SUBUNITS, EXPRESSION AND USES THEREOF

(71) Applicant: Hawaii Biotech, Inc., Honolulu, HI (US)

(72) Inventors: David E. Clements, Honolulu, HI (US); James T. Senda, Honolulu, HI (US); Jaime S. Horton, Honolulu, HI (US)

(21) Appl. No.: 18/024,538

(22) PCT Filed: Sep. 1, 2021
(86) PCT No.: PCT/US2021/048750

§ 371 Date: Mar. 3, 2023

Related U.S. Application Data
(60) Provisional application No. 63/225,783, filed on Jul. 26, 2021.
(60) Provisional application No. 63/075,022, filed on Sep. 4, 2020.

(51) Int. Cl. C07K 14/005; C12N 15/85; A61K 39/39; A61K 39/215; A61P 31/14

(52) U.S. Cl. CPC C07K 14/005; C12N 15/85; A61K 39/39; A61K 39/215; A61P 31/14; C12N 2770/20022; C12N 2770/20034; A61K 2039/55572

(57) ABSTRACT

The present invention is directed to the expression and secretion recombinant SARS-CoV-2 spike protein subunits. Various subunits have been designed and expressed as secreted products into the culture medium of transformed insect cell lines. The design of subunits is focused on the production of products that provide the ability to induce focused immune responses without inducing immune enhancing responses. The expressed and purified products are suitable as vaccine candidates to protect against disease caused by SARS-CoV-2.

### CROSS REFERENCE TO RELATED APPLICATIONS

This application claims the benefit of U.S. Provisional Patent Application Nos. 63/225,783, filed Jul. 26, 2021 and 63/075,022, filed Sep. 4, 2020, which are herein incorporated by reference in their entirety.

### STATEMENT REGARDING FEDERALLY SPONSORED RESEARCH

This invention was made with government support under contract numbered 3R44AI118017-0351 (NIH). The government has certain rights in the invention.

### BACKGROUND OF THE INVENTION

### Field of the Invention

The invention relates generally to the design of optimized SARS-CoV-2 virus spike glycoprotein genes and expression thereof and more specifically to the development of a COVID-19 vaccine.

### Background Information

In the past 18 years there have been 3 significant Coronavirus outbreaks that have greatly impacted global health. First, there was the Severe Acute Respiratory Syndrome (SARS) in 2002, then the Middle East Respiratory Syndrome (MERS) in 2012, and most recently the 2019 novel Coronavirus (2019 nCoV). The 2019 nCoV is now officially named SARS-CoV-2 and the disease it causes is referred to as COVID-19. On Mar. 11, 2020, as a result of the rapid global spread of the virus, the WHO declared that COVID-19 had reached pandemic levels. As of Jun. 10, 2021, there are now greater than 175,000,000 confirmed cases worldwide, and the United States has over 33,400,000 confirmed cases, the most reported by any single country.

The emergence of novel coronaviruses necessitates the need to develop effective medical countermeasures to effectively respond to the public health threat posed by these viruses. Vaccines are one of the most effective methods to combat infectious disease threats such as that posed by the SARS-CoV-2.

Currently, there is no licensed vaccine for SARS-CoV-2; however, three SARS-CoV-2 vaccines have received Emergency Use Authorization (EUA) from the U.S. FDA as of April 2021. These include the mRNA-based Pfizer-BioNTech BNT162b2 and Moderna mRNA-1273 vaccines, as well as Janssen's Ad26.COV2.S adenovirus vector-based vaccine. According to the World Health Organization there are currently 276 vaccine candidates in development (WHO 27apr.2021), underscoring the ongoing need for continued vaccine development. Ninety-two of these vaccine candidates are currently in various stages of clinical trials and the other 184 vaccine candidates are in various stages of preclinical development. These approaches employ methods that include inactivated virus, attenuated virus nucleic acid-based, both mRNA and DNA, viral vectored, VLP based, and recombinant subunit proteins. While any one of these may progress to a viable candidate in the coming months to years, there are knowledge gaps that could impact the ultimate realization of developing a safe and effective SARS-CoV-2 vaccine. First, the selection of ideal antigen(s) is not yet defined. While the spike protein (S) is the leading candidate, it is yet to be determined as to which portion of S is best: full length, ectodomain, S1, or receptor binding domain (RBD) only. Second, preclinical studies with SARS and MERS vaccine candidates have raised safety concerns due to enhancement of disease as a direct result of skewed antibody responses. Third, there are no clear correlates of protection for SARS-CoV-2. While they can be inferred from SARS and MERS studies, correlates still need to be established for SARS-CoV-2. Lastly, there are concerns about the duration of immunity elicited by vaccines. It is critical that these various aspects which will have an impact on the outcome of any vaccine candidate be addressed in the vaccine development process.

In addition to the concerns related to the immune responses elicited by current vaccine candidates, there are also concerns related to each of the platform technologies that are being utilized. These include product stability, product delivery methods, potential for cGMP scale up, and regulatory pathways. For example, mRNA vaccines have unique challenges related to the lipid nanoparticle vehicle employed (stability and reactogenicity/toxicity). While DNA vaccines have ease of manufacturing and scalability in their favor, delivery methods and weak immunogenicity in humans still pose significant challenges. Replication-competent vaccines have advantages of rapid immune responses with a single dose; however, manufacturing processes that rely on viral replication become more pronounced with scale-up and some require more extensive cold chain requirements. For example, the current Merck VSV-based Ebola vaccine requires −60° C. storage conditions. It has also been reported that the mRNA vaccines will require −20° C. storage conditions. If an inactivated vaccine approach is pursued, there will be safety concerns with the scaling of live SARS-CoV-2 and the need for BSL3 containment. Alternative vaccine manufacturing platforms that can avoid some, if not all, of these gaps/challenges are of great value. Recombinant subunit SARS-CoV-2 vaccines provide an opportunity to address many of these concerns. Furthermore, it has become clear that having more than one technology platform/manufacturer for emerging infectious disease is important to ensure adequate vaccine supplies.

The choice of a recombinant protein expression system to employ is dependent on the desired application. The system of choice must meet key criteria such as proper folding and processing, consistency, and productivity (cost effectiveness) of the desired protein product. Insect cell-based expression systems have the potential to meet capacity requirements based on ease of culture, higher tolerance to osmolality and by-product concentrations during large scale culture, and generally higher expression levels.

Recently, the use of expression systems based on insect cells has become more common. These systems provide most of the characteristics desired of eukaryotic systems but have added benefits such as lower cost of goods. Insect cell systems are either based on infection of host cells with insect virus vectors (e.g., baculovirus) or on the generation of stable cell lines by integration of expression plasmids into the genome of the host cells.

The baculovirus expression system (BES) has emerged as the primary insect cell culture system utilized for recombinant protein expression. This system is based on the use of vectors derived from the insect viruses known as baculovirus. These vectors are used to generate recombinant viruses that encode the desired protein product. The recombinant viruses are used to infect host insect cells that then express the desired recombinant proteins. While there are advantages to this system in regard to ease of cloning and “time to product”, there are also several disadvantages. The primary challenge in the use of BES is that it is based on the viral infection of the host cells. This results in cellular lysis and cell death 72-96 hrs post infection. As a result, during the late stages of infection the processing machinery of the insect cells is compromised to the extent that the processing of the desired product is also compromised. This limits the time that the cells can produce product and possibly more importantly leads to altered forms of the product being produced. Furthermore, the lysis of cells releases large amounts of cellular debris and enzymes that can impact the quality of the desired product.

The use of stably transformed insect cells for the expression of recombinant proteins is an alternative to the use of BES. Expression systems based on stably transformed insect cell lines are non-lytic and provide for steady long-term production of secreted products that require proper folding and post translational modifications. The secretion of the product into the culture medium provides a cleaner starting material for the purification process and allows for the final protein product to be purified with basic methods. This leads to products that are of higher quality.

The development of a recombinant subunit vaccine for SARS-CoV-2 requires the selection of appropriate gene sequences from the SARS-CoV-2 genome that encode proteins that are the target of neutralizing antibodies (nAbs). Like other members of the coronavirus family, the spike glycoprotein of SARS-CoV-2 is the primary target of nAbs. In addition to selection of an appropriate SARS-CoV-2 gene sequence, efforts to optimize the expression of the selected gene sequences is also desirable to enhance the ability to effectively express the selected sequences such that the resultant products are soluble, stable and conformationally relevant.

The combination of multiple optimizations directed at different aspects of a protein's gene sequence and or structure in an appropriate manner such that an additive benefit is achieved can further enhance the utility of the optimized protein product. For SARS-CoV-2 S glycoprotein subunit proteins, examples exist of expression of various truncated products as soluble recombinant proteins which indicate various levels of optimization; however, these examples are only reporting moderate expression levels and employ limited approaches to optimization. Given the need for a vast number of vaccine doses to combat the SARS-CoV-2 pandemic, efforts to further the optimal expression of high levels of SARS-CoV-2 S glycoprotein subunits are needed. There are no examples of where multiple components are optimized to further enhance the expression and secretion of soluble SARS-CoV-2 S glycoproteins in insect cells. Therefore, the technical problems to be solved are: (1) identification of translational, posttranslational, or structural components of the spike protein or associated components that when optimized result in improved expression levels and potentially enhance structural quality of the protein such that it is a more potent immunogen; (2) the design of synthetic components where possible to aid in the optimization; and (3) determining the optimal truncations points to increase in the productivity and quality of protein expression. Further improvements in the expression of the SARS-CoV-2 S glycoprotein S protein subunits in insect cells provide for effective immunogens at an improved cost of goods which would bolster the ability to manufacture recombinant proteins suitable for use in vaccines to combat the threat posed by SARS-CoV-2. The use of such improvements could also be applied to other members of the coronavirus family.

### SUMMARY OF THE INVENTION

The invention provides optimized expression of soluble recombinant SARS-CoV-2 S subunit proteins that result in high levels of expression of a native-like or biologically relevant proteins; and is therefore, an effective immunogen for the production of nAbs. Specifically, the invention is directed to expression of the optimized SARS-CoV-2 S gene sequences when Drosophila melanogaster S2 cells are used as the host cell.

The invention also provides methods for utilizing the products encoded by the optimized SARS-CoV-2 S gene sequences in vaccine formulations for protecting against disease caused by infection with SARS-CoV-2.

In one embodiment, the invention provides an isolated nucleic acid sequence selected from SEQ ID NO: 1, 2, 3, 4, 5 and 6.

In another embodiment, the invention provides an isolated amino acid sequence encoded by a nucleic acid sequence selected from SEQ ID NO: 1, 2, 3, 4, 5 and 6.

In one aspect, the amino acid sequence includes SEQ ID NO: 9, 10, 11, 12, 13 or 14.

In an additional embodiment, the invention provides an expression vector including a nucleic acid sequence encoding a SARS-CoV-2 spike (S) protein, wherein the nucleic acid sequence includes SEQ ID NO: 1, 2, 3, 4, 5 or 6.

In one aspect, the vector is a Drosophila melanogaster expression vector. In some aspects, the vector has a nucleic acid sequence including SEQ ID NO: 7. In another aspect, the SARS-CoV-2 S protein has an amino acid sequence including SEQ ID NO: 9, 10, 11, 12, 13 or 14.

In one embodiment, the invention provides a method of producing a protein in vitro including an expression vector with an operably-linked nucleic acid sequence encoding a SARS-CoV-2 spike (S) protein, wherein the nucleic acid sequence includes SEQ ID NO: 1, 2, 3, 4, 5 or 6 using Drosophila melanogaster cells and culturing the cells under conditions to produce the protein.

In one aspect, the Drosophila melanogaster cells are Schneider 2 (S2) cells.

In another embodiment, the invention provides a vaccine composition including (a) an effective amount of a SARS-CoV-2 spike (S) protein, wherein S protein is encoded by a nucleic acid sequence including SEQ ID NO: 1, 2, 3, 4, 5 or 6 and (b) an effective amount of an adjuvant selected from the group consisting of GPI-0100, synthetic lipid A (SLA) in a stable oil-in water emulsion (SE) (SLA-SE), QS21, QS21 combined with SLA to form a liposome formulation (SLA-LSQ), and QS21+CpG.

In one aspect, the S protein is a 51 subunit protein. In some aspects, the 51 subunit protein is encoded by a nucleic acid sequence of SEQ ID NO: 3. In another aspect, the adjuvant is SLA-SE. In one aspect, the vaccine composition includes the 51 subunit protein with the amino acid sequence of SEQ ID NO: 10 and the adjuvant SLA-SE. In another aspect, the 51 subunit protein is recombinantly produced and expressed in insect host cells. In some aspects, the vaccine composition further includes a pharmaceutically acceptable excipient or carrier.

In one embodiment, the invention provides a method of preventing SARS-CoV-2 entry into a target cell including contacting a subject with the target cell of SARS-CoV-2 with a therapeutically or prophylactically effective amount of composition including (a) an effective amount of a SARS-CoV-2 spike (S) protein wherein the S protein is encoded by a nucleic acid sequence including SEQ ID NO: 1, 2, 3, 4, 5 or 6, and (b) an effective amount of an adjuvant, thereby preventing SARS-CoV-2 entry into the cell.

In another embodiment, the invention provides a method of stimulating a protective immune response in a subject including administering to the subject a therapeutically or prophylactically effective amount of composition including (a) an effective amount of a SARS-CoV-2 spike (S) protein, wherein the S protein is encoded by a nucleic acid sequence including SEQ ID NO: 1, 2, 3, 4, 5 or 6, and (b) an effective amount of an adjuvant, thereby stimulating a protective immune response in the subject.

In one aspect, the immune response is a balanced immune response. In another aspect, a balanced immune response is characterized by an IgG2a:IgG1 ratio that is equal or greater than 1.

In an additional embodiment, the invention provides a method of inhibiting a SARS-CoV-2 infection in a subject including administering to the subject a therapeutically or prophylactically effective amount of composition including (a) an effective amount of a SARS-CoV-2 spike (S) protein, wherein the S protein is encoded by a nucleic acid sequence including SEQ ID NO: 1, 2, 3, 4, 5 or 6, and (b) an effective amount of an adjuvant, thereby inhibiting SARS-CoV-2 infection.

In another embodiment the invention provides a method of inhibiting transmission of a SARS-CoV-2 infection by a subject including administering to the subject a therapeutically or prophylactically effective amount of composition including (a) an effective amount of a SARS-CoV-2 spike (S) protein, wherein S protein is encoded by a nucleic acid sequence including SEQ ID NO: 1, 2, 3, 4, 5 or 6, and (b) an effective amount of an adjuvant selected from the group consisting of GPI-0100, synthetic lipid A (SLA) in a stable oil-in water emulsion (SE) (SLA-SE), QS21, QS21 combined with SLA to form a liposome formulation (SLA-LSQ), and QS21+CpG, thereby inhibiting transmission of a SARS-CoV-2 infection.

In one aspect, the S protein is a 51 subunit protein. In some aspects, the 51 subunit protein is encoded by a nucleic acid sequence of SEQ ID NO: 3. In another aspect, the adjuvant is SLA-SE. In one aspect, the vaccine composition including the 51 subunit protein with the amino acid sequence of SEQ ID NO: 10 and the adjuvant SLA-SE. In another aspect, the vaccine induces the production of nAbs in the subject. In some aspects, the nAbs prevent the binding of a SARS-CoV-2 to a target cell and/or target receptor. In various aspects, the target receptor is an ACE2 receptor. In one aspect, administering includes injecting two doses to the subject at a 3-weeks interval. In another aspect, administering includes injecting intramuscularly. In some aspects, a dose comprises about 0.5-50 μg of purified S protein. In other aspects, administering the vaccine to the subject increases the subject survival. In some aspects, administering the vaccine prevents the development of COVID-19 disease in the subject.

Another aspect of the present invention is to provide a method to elicit an immune response that provides protection against disease caused by SARS-CoV-2 infection. The method includes administering to a subject in need thereof a composition that includes a soluble spike protein subunit expressed and secreted by an expression vector that includes a codon optimized DNA sequence that includes SEQ ID NO: 1, 2, 3, 4, 5 or 6.

In one aspect, the composition includes an adjuvant to enhance the immune response.

Other aspects and advantages of the invention will be apparent from the following description and the appended claims.

### BRIEF DESCRIPTION OF THE DRAWINGS

FIG. 1. SARS-CoV-2 Spike Protein Linear Map. Schematic of SARS-CoV-2 Spike protein primary structure with the various domains labeled, SS signal sequence; S2′, S2′ protease cleavage site; FP, fusion peptide; HR1, heptad repeat 1; CH, central helix; CD, connector domain; HR2, heptad repeat 2; TM, transmembrane domain; CT, cytoplasmic tail. Domains that were excluded from the ectodomain expression construct or could not be visualized in the final map are colored white. Adapted from Wrapp et al (2020).

FIG. 2. SARS-CoV-2 Spike Protein Structure. Crystal structure of single pre-fusion protomer from SARS-CoV-2 Spike protein trimer with the RBD in the up conformation is shown. The main domains are labeled, 51, S2, and 51 subdomains, NTD N-terminal domain; RBD receptor binding domain. Adapted from Wrapp et al (2020).

FIG. 3. Alignment of the amino acid sequences from all 2019 nCoV Spike protein subunits expressed in Drosophila S2 cells relative to the 2019 nCoV Spike full length protein sequence (SEQ ID NO 8). Modifications relative to the native sequence are indicated in bold and underlined sequences.

FIG. 4. Diagram of the SARS-COV-2 spike subunit proteins expressed in Drosophila S2 cells relative to the full-length spike protein. Spike 1-1273 is SEQ ID NO: 8 (aa 1-1273); Ecto aa 14-1147 of SEQ ID NO: 8 (SEQ ID NO: 1); Ecto-F 14-1147 of SEQ ID NO: 8 with L-Fold (SEQ ID NO: 2); 51 aa 14-594 of SEQ ID NO: 8 (SEQ ID NO: 3); RBD aa 318-594 of SEQ ID NO: 8 (SEQ ID NO: 4); RBD-F aa 318-594 of SEQ ID NO: 8 with L-Fold (SEQ ID NO: 5); and NTD aa 14-305 of SEQ ID NO: 8 (SEQ ID NO: 6).

FIG. 5. The wild type Wuhan-Hu-1 spike nucleotide sequence (SEQ ID NO: 15) along with translation (SEQ ID NO: 8).

FIG. 6. Expression of nCoV Spike Ectodomain. Coomassie stained gel and Western Blot with unconcentrated culture medium from parental S2 cell lines expressing the nCoV-S-S SDQ-Ecto-CO, nCoV-S-SQ-Ecto-CO and nCoV-S-WT-Ecto. Arrows indicate the position of the S-Ecto protein.

FIG. 7. Expression of nCoV-S-S1-CO. Coomassie stained gel and Western Blot with unconcentrated culture medium of S2 cell lines compared to purified protein. Arrow indicates the position of the nCoV-S-S1 protein.

FIG. 8. Binding of recombinant hACE2-His-tagged protein to expressed recombinant SARS-CoV-2 spike proteins. Two different lots of each the expressed SARS-CoV-2 spike protein subunits, RBD, 51, and Ecto, were coated on ELISA plates and detected with recombaint hACE2 protein. RBD-Mo lot 1009 and 51 lot 1014 show strong binding of hACE2 protein indicating structural and functional integrity.

FIG. 9. Expression of nCoV-S-RBD-CO. Coomassie stained gel and Western Blot with unconcentrated culture medium of S2 cell lines compared to purified protein. Arrow indicates the position of the nCoV-S-RBD protein.

FIG. 10. Expression of nCoV-S-RBD-CO-foldon and nCoV S SSDQ Ecto CO-foldon. Coomassie stained gel and Western Blot with unconcentrated culture medium of S2 cell lines compared to subunits lacking the foldon domain and purified protein. Arrows indicates the position of the nCoV-S-RBD-foldon and nCoV-S-Ecto-foldon protein.

FIG. 11. Expression of nCoV-S-NTD-CO. Coomassie stained gel and Western Blot with unconcentrated culture medium of S2 cell lines compared to purified protein. Arrow indicates the position of the nCoV-S-NTD protein.

FIG. 12. Immunogenicity of nCoV-S-RBD-foldon in mice. ELISA analysis of serum after 2 and 3 doses. The dilution of serum that results in half maximal binding (EC50) is reported as geometric mean (GMT) for each group.

FIG. 13. Immunogenicity of nCoV-S-RBD-foldon in mice. Micro-neutralization analysis of serum post-dose 2 (PD2) and post-dose 3 (PD3). The dilution of serum that results in 50% neutralization (MN₅₀) is determined for each serum sample. The MN₅₀ geometric mean (GMT) for each group is reported.

FIG. 14. Immunogenicity of nCoV-S-RBD-foldon in mice. RBD blocking analysis of serum post-dose 2 (PD2) and post-dose 3 (PD3). The percent blocking (reduction) for each of serum samples in each group are plotted as reciprocal dilutions.

FIGS. 15A-15B. Results of blocking assay and the MN assay for the serum samples for study M-001. FIG. 15A. Blocking assay results are reported as geometric mean (GMT) of the EC₅₀ of blocking for individual serum samples. FIG. 15B. Micro-neutralization (MN₅₀) titers are reported for serum pools for each group tested in duplicate.

FIG. 16A-16B. Results of blocking assay and the MN assay for the serum samples for study M-002. FIG. 16A. Blocking assay results are reported as geometric mean (GMT) of the EC₅₀ of blocking for individual serum samples. FIG. 16B. Micro-neutralization (MN₅₀) titers are reported for serum pools for each group tested in duplicate.

FIG. 17. ELISA results reported as IgG2a/IgG1 ratio for Immunogenicity Study M-002.

FIG. 18. IgG2a/IgG1 ratios for select groups from Immunogenicity Studies M-000, M-001, and M-002. P values determined using one-way ANOVA test as compared to the Alum Group. If P value is not shown then it was not significant, otherwise **=P<0.01, ***=P<0.001, ****=P<0.0001.

FIGS. 19A-19B. Results for the M-003 challenge study: Percent of body weight change post challenge and a summary table of results that includes, pre-challenge MN₅₀ results and percent survival for each group.

FIG. 20A-20B. Results for the M-005 challenge study: Percent of body weight change post challenge and a summary table of results that includes, pre-challenge and post-challenge MN₅₀ results, and percent survival for each group.

### DETAILED DESCRIPTION OF THE INVENTION

The present invention is based on the seminal discovery that truncated and codon-optimized nucleic acid sequences encoding novel SARS-Cov-2 spike protein subunits can be expressed and formulated in vaccine compositions comprising such novel SARS-Cov-2 spike protein subunits, and for methods of use thereof.

Before the present compositions and methods are described, it is to be understood that this invention is not limited to particular compositions, methods, and experimental conditions described, as such compositions, methods, and conditions may vary. It is also to be understood that the terminology used herein is for purposes of describing particular embodiments only, and is not intended to be limiting, since the scope of the present invention will be limited only in the appended claims.

As used in this specification and the appended claims, the singular forms “a”, “an”, and “the” include plural references unless the context clearly dictates otherwise. Thus, for example, references to “the method” includes one or more methods, and/or steps of the type described herein which will become apparent to those persons skilled in the art upon reading this disclosure and so forth.

All publications, patents, and patent applications mentioned in this specification are herein incorporated by reference to the same extent as if each individual publication, patent, or patent application was specifically and individually indicated to be incorporated by reference.

Unless defined otherwise, all technical and scientific terms used herein have the same meaning as commonly understood by one of ordinary skill in the art to which this invention belongs. Although any methods and materials similar or equivalent to those described herein can be used in the practice or testing of the invention, it will be understood that modifications and variations are encompassed within the spirit and scope of the instant disclosure. The preferred methods and materials are now described.

The invention provides optimized SARS-CoV-2 spike gene sequences for expression of soluble and stable spike protein subunits that are composed of contiguous sequences that have been codon optimized, contain an optimized signal peptide cleavage site, and have optimized N-terminal and C-terminal ends that enhance expression and stability of the expressed spike subunit proteins. The optimized gene sequences are inserted into a Drosophila S2 cell expression vector which drives the expression of high levels of high-quality SARS-CoV-2 spike subunit proteins in S2 cells that have been stably transformed with the expression vectors carrying the optimized gene sequences. The use of the optimized gene sequences results in an increase in the productivity and quality of the expressed SARS-CoV-2 spike subunit proteins. The enhanced expression of the SARS-CoV-2 spike subunit proteins provides for production of effective immunogens at an improved cost of goods which can bolster the ability to manufacture recombinant proteins suitable for use in vaccines to combat the spread of the SARS-CoV-2.

SARS-CoV-2 Spike Optimized Proteins, Polynucleotides Encoding them and Expression Vectors Thereof

In one embodiment, the invention provides an isolated nucleic acid sequence selected from SEQ ID NO: 1, 2, 3, 4, 5 and 6. As used herein, the term “nucleic acid” or “oligonucleotide” refers to polynucleotides such as deoxyribonucleic acid (DNA) or ribonucleic acid (RNA). Nucleic acids include but are not limited to genomic DNA, cDNA, mRNA, iRNA, miRNA, tRNA, ncRNA, rRNA, and recombinantly produced and chemically synthesized molecules such as aptamers, plasmids, anti-sense DNA strands, shRNA, ribozymes, nucleic acids conjugated and oligonucleotides. According to the invention, a nucleic acid may be present as a single-stranded or double-stranded and linear or covalently circularly closed molecule. A nucleic acid can be isolated. The term “isolated nucleic acid” means, that the nucleic acid (i) was amplified in vitro, for example via polymerase chain reaction (PCR), (ii) was produced recombinantly by cloning, (iii) was purified, for example, by cleavage and separation by gel electrophoresis, (iv) was synthesized, for example, by chemical synthesis, or (vi) extracted from a sample. A nucleic acid might be employed for introduction into, i.e. transfection of, cells, in particular, in the form of RNA which can be prepared by in vitro transcription from a DNA template. The RNA can moreover be modified before application by stabilizing sequences, capping, and polyadenylation.

The terms “sequence identity” or “percent identity” of polynucleotides or polypeptides are used interchangeably herein. To determine the percent identity of two polypeptide molecules or two polynucleotide sequences, the sequences are aligned for optimal comparison purposes (e.g., gaps can be introduced in the sequence of a first polypeptide or polynucleotide for optimal alignment with a second polypeptide or polynucleotide sequence). The amino acids or nucleotides at corresponding amino acid or nucleotide positions are then compared. When a position in the first sequence is occupied by the same amino acid or nucleotide as the corresponding position in the second sequence, then the molecules are identical at that position. The percent identity between the two sequences is a function of the number of identical positions shared by the sequences (i.e., % identity=number of identical positions/total number of positions (i.e., overlapping positions)×100). In some embodiments the length of a reference sequences (e.g. SEQ ID NOs:1-6) aligned for comparison purposes is at least 80% of the length of the comparison sequence, and in some embodiments is at least 90% or 100%. In an embodiment, the two sequences are the same length.

Ranges of desired degrees of sequence identity are approximately 80% to 100% and integer values in between. Percent identities between a disclosed sequence and a claimed sequence can be at least 80%, at least 85%, at least 90%, at least 95%, at least 96%, at least 97%, at least 98%, at least 99%, at least 99.5%, or at least 99.9%. In general, an exact match indicates 100% identity over the length of the reference sequences (e.g., SEQ ID NOs:1-6).

Polypeptides and polynucleotides that are about 85, 86, 87, 88, 89, 90, 91, 92, 93, 94, 95, 96, 97, 98, 99, 99.5% or more identical to polypeptides and polynucleotides described herein are embodied within the disclosure. For example, a polynucleotide can have 80%, 85%, 90%, 91%, 92%, 93%, 94%, 95%, 96%, 97%, 98%, 99% or more identity to SEQ ID NOs:1-6.

The term “gene sequence” refers to a sequence of DNA that is transcribed into an RNA molecule that may function directly or be translated into an amino acid chain.

The term “optimized” refers to sequences that were derived from naturally occurring sequences and have been altered to enhance their functions.

The term “codon optimized” refers to a nucleic acid coding region that has been adapted for expression in the cells of a given host by replacing at least one, or more than one, or a significant number, of codons with one or more codons that are more frequently used in the genes of that host.

One aspect of the present invention is to provide a codon optimized DNA sequence encoding a SARS-CoV-2 S protein subunit. The expression of the DNA sequence results in secretion of a soluble S protein subunit in the culture medium.

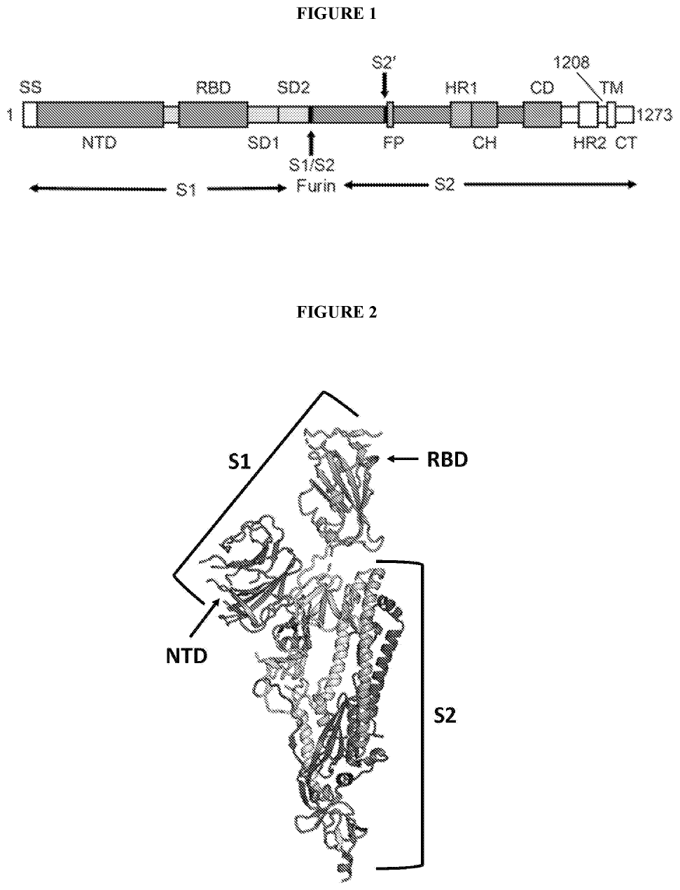

The coronavirus (CoV) genome encodes four major structural proteins: nuclear, membrane, small envelope, and spike (S). The S protein is the major viral surface protein and mediates viral entry. Recent studies indicate that human angiotensin-converting enzyme 2 (hACE2) protein is the major cellular receptor for the S protein for SARS-CoV-2 (Wall et al, 2020). The S protein of SARS-CoV and SARS-CoV-2 has also been shown to be the target of virus nAbs (He et al, 2006; Wall et al, 2020). The S protein is a class 1 fusion protein related to HIV, influenza and Ebola. The S protein is divided into the S1 and S2 domains. The S1/S2 junction is delineated by a furin protease cleavage site. S1 contains the receptor binding domain (RBD) and the S2 contains the fusion peptide, 2 helical domains, transmembrane domain, and cytoplasmic tail. FIG. 1 shows the primary structure of the S protein and the various features. The S protein forms a homotrimer that make up the spikes on the surface of the virus. The crystal structure of the SARS-CoV-2 S glycoprotein has recently been solved (Wall et al, 2020; Wrapp et al, 2020). FIG. 2 shows a single protomer of the S trimer.

As indicated above, the S glycoprotein has been determined to be the target of virus nAb; therefore, it is an obvious choice for vaccine development. There are several factors that need to be considered in an effort to define which portions of the S glycoprotein comprises a suitable vaccine candidate when expressed as a recombinant subunit. First, the expression of a soluble product requires the exclusion of the transmembrane domain, thus the need to define an appropriate carboxy terminal truncation point. Second, the S glycoprotein contains a furin protease cleavage site which can impact the integrity of an expressed protein product. Third, as the goal of a vaccine candidate based on the S glycoprotein is to promote the production of nAb in immunized subjects, the relevant regions and structural constraints need to be considered. Fourth, while the focus is on regions of spike that elicit nAbs, there is also benefits in excluding certain segments of the protein that may distract form the ability to elicit nAbs. Fifth, there are concerns that various portions of the S glycoprotein can contribute to immune enhancement or immunopathology and, therefore, selection of the portions of the protein that avoid this potential is preferred. In order to develop SARS-COV-2 S glycoprotein subunit vaccine candidate, it is important to consider the factors above so that a suitable vaccine candidate can be produced. Various methods can be applied to enhance the quality and productivity of expressed recombinant S glycoprotein subunit proteins such that they will result in effective vaccine candidates.

While there are examples of coronavirus spike gene sequences being expressed, there are no clear examples of expression-enhancing sequence optimization, as most examples utilize the naturally occurring (wild type) sequences of the parent viruses. In an effort to produce selected recombinant proteins as vaccine candidates, it is important to optimize conditions to express these proteins at high levels and in proper conformations such that they serve as effective vaccines. Various means exist to optimize expression. However, each selected sequence requires several rounds of experimentation to determine which methods or combination of methods will result in the most effective expression in a given expression system while maintaining the appropriate native, or biologically relevant characteristics. These efforts to optimize selected sequences are a means to enhance the immunogenic potential to induce a nAb response, in this case, efforts are focused on the SARS-CoV-2 spike gene sequence.

In the course of expressing optimized gene sequences for the production of desired recombinant protein subunits, it is important to utilize biologically functional methods to assess the potential of the expressed products to assess to potential of the products. For vaccine candidates targeting viral diseases, the primary criteria are to assess the ability of the proteins to elicit potent and specific antibody responses when used to immunize animals. The most basic assay is the Enzyme Linked Immuno-Sorbent Assay (ELISA) which measures total antibody binding to a specific target. This provides a general measure of the strength of the immune response following a course of immunization. A more specific assay is one in which the ability of serum to neutralize virus from infecting cells in culture. There are various forms of these assays, based on how viral infection of the cells are measured. One form of the assay is the Micro-Neutralization (MN) test which monitors cytopathic effect (CPE) on the cells following the addition of virus and serum mixture. Lastly, when the cell receptor and the receptor binding site on the viral proteins are known and recombinant versions are available, assays that measure the ability to block protein-protein interaction (binding) can be set up. In the case of SARS-CoV-2, the receptor binding domain has been defined and the host cell receptor is known to be ACE2. The use of these assays provide can provide strong evidence that properly designed and expressed recombinant protein subunits are potential vaccine candidates.

In efforts to express proteins, problems are often encountered with virus envelope sequences that impact the appropriate native or biologically relevant characteristics which can hamper optimal expression. These problems include non-optimal truncations that define the amino-terminus and the carboxy-terminus of the expressed product, poor or ineffective post-translational processing, and poor matching of the native codon usage with the codon usage of the selected expression system's host cells.

The efficiency of heterologous protein expression in eukaryotic systems is dependent on many factors, such as promoter and associated regulatory elements, transcription initiation sequences, and poly-adenylation signals. As the expression vectors used in a typical system are optimized for the given host cell utilized, the optimization of the gene sequence of interest is often of great importance to ensure optimal expression of the desired protein product. This is typically done by adaptation of the codon usage of the gene sequence to the typical codon usage of the host cells. While the gene sequence is altered through codon optimization, the amino acid sequence of the encoded protein in not modified through the optimization process (Gustafsson et al, 2004). Basic codon usage optimization involves substituting rare codons in the target gene sequence to ones used more frequently by the host cells. Alternatively, the entire gene sequence can be altered to be in line with the codon usage of the host cells used to express the desired product. With the current efficiency of de novo gene synthesis, the latter approach has become the preferred method of codon optimization. As the expression of heterologous proteins is an important part of the biotechnology industry, methods such as codon optimization are often useful in improving expression levels.

Most proteins that are secreted from cells contain an N-terminal signal sequence that directs the protein into the cell's secretion pathway. Optimization of internal secretion signal or signal peptide sequences that interact with the endoplasmic membrane to initiate the secretion process has the potential to increase the efficiency of processing and hence an increase in protein expression. The eukaryotic signal sequence has been divided into three structural regions, basic, hydrophobic, and polar, starting from the N-terminus and proceeding to the C-terminus respectively (von Heijne, 1986 and Bendtsen et al 2004). Over the years, numerous secretion signals have been identified and used to direct the secretion of recombinant proteins. Although many different signal sequences have been used and shown to be functional, few studies have been reported that define optimal sequences for a given cell type. The general characteristics and rules related to the three structural regions are well established, as detailed by von Heijne (1986) and by Bendtsen et al (2004), however, little comparative experimental data exist as to what constitutes an optimal secretion signal in a given expression system or a given heterologous protein being expressed. Most published reports deal with the characterization and optimization of gram positive bacterial or yeast secretion signals (Le Loir et al, 2005 and Hofmann and Schultz, 1991). One report that describes the optimization of the IL-2 secretion signal clearly demonstrates the benefits of optimization (Zhang et al, 2005). While the secretion signal peptide is important for optimal expression of secreted proteins, the signal peptidase cleavage site is also an important part in the secretion process. In designing heterologous expression and secretion of selected protein sequences it is important to ensure the signal peptidase cleavage site remains optimal.

Many eukaryotic proteins are modified by N-linked glycosylation (asparagine-linked). The of number of glycosylation sites and the efficiency of glycosylation by the enzyme oligosaccharyltransferase can vary for each protein expressed and can be based on a number of factors. This can influence its expression and function. N-Linked glycosylation usually occurs at the Asn residues in the Asn-X-Ser/Thr motif, where X is any amino acid except for Pro. However, many Asn-X-Ser/Thr sequences are not glycosylated or are glycosylated inefficiently (Mellquist et al, 1998). Inefficient glycosylation at one or more Asn-X-Ser/Thr sequences in a protein results in the production of heterogeneous glycoprotein products. The work of Mellquist et al has revealed that the amino acid at the Y position (amino acid residue immediately following the Ser or Thr residue) is an important determinant of core glycosylation efficiency. This provides an example of a means to optimize the glycosylation efficiency of proteins expressed in heterologous systems.

The surface proteins of enveloped viruses form multimeric configurations. The spike protein on the surface of coronaviruses is defined as a class 1 fusion protein as it forms a homotrimer. These trimeric structures are anchored in the virus membrane shell by the transmembrane (TM) domains. Expression of the spike protein ectodomain (lacking the TM) results in monomers. Various methods are available to drive the formation of multimeric forms of expressed proteins. In general, these methods involve the fusion of protein domains to the protein of interest. These protein domains can promote the formation of multimeric states of the protein. A commonly used protein domain used to promote trimer formation consist of a 29 amino acid sequence that is derived from the bacteriophage T4 fibritin protein sequence. This is referred to as the “foldon” sequence. The foldon sequence is located at the C-terminus of the fibritin protein, and naturally brings together three monomers of fibritin via non-covalent bonding to form a trimeric molecule. The use of the foldon domain provides a means to drive the formation of trimeric molecules when fused to the protein of interest.

As described above, various structural aspects are required for optimal protein expression. When selected protein sequences are chosen for expression in a selected cell system, these structural aspects can be altered to enhance the expression level of the desired protein product. Alternatively, modification of internal protein sequences to enhance selected epitopes or to remove selected epitopes can be employed to create a more desired product, as in, a protein product that has an enhanced immunogenic potential. As an example, epitopes in the dengue E protein that are believed to generate flavivirus cross-reactivity antibody responses were altered to reduce the potential of immune enhancement (Hughes et al, 2012). These included the immunodominant B cell epitope of the fusion peptide and domain III epitopes. The SARS-CoV S glycoprotein provides another example where the peptide 597-603 has been identified as a B-cell epitope that induces antibodies that enhance infection. (Wang et al, 2016). This same location in the spike protein of SARS-CoV-2 has been identified to contain a mutation, Asp 614 to Gly, that results in higher virus titers, but also may have an immunological role (Korber et al, 2020).

The crystal structure of the SARS-CoV-2 S glycoprotein has recently been solved (Wall et al, 2020; Wrapp et al, 2020); however, the optimal gene sequence for the expression of S proteins subunits in eukaryotic host cell expression systems are not currently well defined. Current technology and methods provide the potential to assemble gene sequences and make modifications to internal sequences and define new end points that can lead to improvements in structure and function. While the potential exists to make such modifications, it is common knowledge that not all attempts to do so result in success. The modifications or attempts to optimize that work with one protein and in a given expression system do not always work on other proteins or in other expression systems. For example, the removal of the first 58 amino acids from the N-terminus of the West Nile envelope protein ectodomain abolishes expression. In another example, the expression of the C-terminal domain (domain III) as of the West Nile or Tick-borne encephalitis envelope protein is easily expressed, however, these subunit proteins are suboptimal in their ability to prime functional immune responses, though it is not obvious why this occurs. In an example specific to SARS-CoV-2, an attempt to express the spike protein subunit NTD (SEQ. ID. NO: 14), resulted in multiple forms of the NTD that were misfolded and were heterogenous in the extent of glycosylation (Example 5). Therefore as illustrated by these examples, a systematic evaluation is required to determine the potential of various efforts to modify and optimize a given gene sequence such that high levels of high-quality heterologous protein are expressed and that such alterations do not negatively impact the desired functional attributes.

In the biotechnology field, the ability to efficiently produce recombinant proteins at a favorable cost of goods is a key to success. In order to achieve this goal for a particular protein, in this case the SARS-CoV-2 S glycoprotein using the Drosophila S2 cell expression system, further experimentation is required to define the parameters that result in optimal expression of a high-quality protein product.

The optimized SARS-CoV-2 gene sequences for expression of soluble and stable subunit proteins are composed of a spike gene sequence that has been codon optimized, that contains an optimized signal peptidase cleavage site, and that has selected amino and carboxy truncation points that enhance expression and stability of the S subunit proteins. The optimized gene sequence is contained in an expression vector for use in Drosophila S2 cells. The codon optimization of the gene sequence is designed for optimal expression in Drosophila S2 cells. The optimized signal peptidase cleavage site is utilized to result in effective post-translational processing at the N-terminus of the subunits. The end points of the gene sequences have been selected to produce optimal N-terminal and C-terminal ends for the spike protein subunits which help to stabilize the expressed subunits. The foldon trimerization domain is added to the C-terminus to further stabilize expressed spike protein subunits. The combination of the optimization methods has resulted in unique gene sequences that provide for the expression of soluble SARS-CoV-2 S proteins at high levels and with enhanced stability. These improved SARS-CoV-2 S protein subunits are suitable for use as vaccine candidates to protect against disease caused by SARS-CoV-2 infection.

The optimized SARS-CoV-2 S gene sequences of the present invention are capable of high-level expression and secretion of the encoded S subunit proteins into the culture medium of transformed S2 cells. Specifically, the described SARS-CoV-2 S gene sequences have been optimized for 1) codon usage in Drosophila S2 cells, 2) a synthetic, optimized secretion signal processing site, and 3) N-terminal and C-terminal truncation points that add stability and function to the expressed products.

In one aspect, the codon optimized DNA sequence encodes the ectodomain with the optimized signal peptidase cleavage site and includes SEQ ID NO: 1.

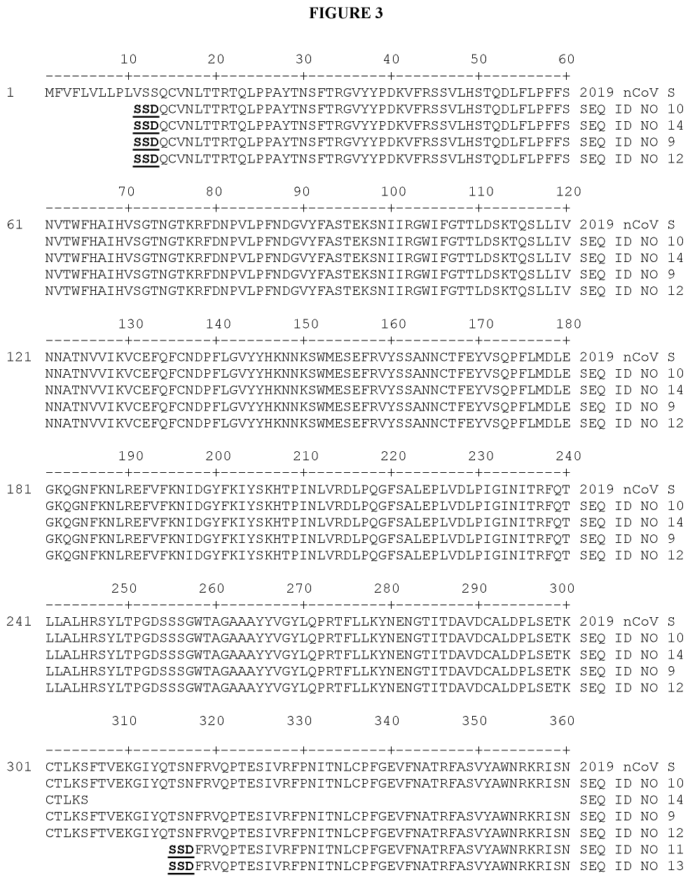

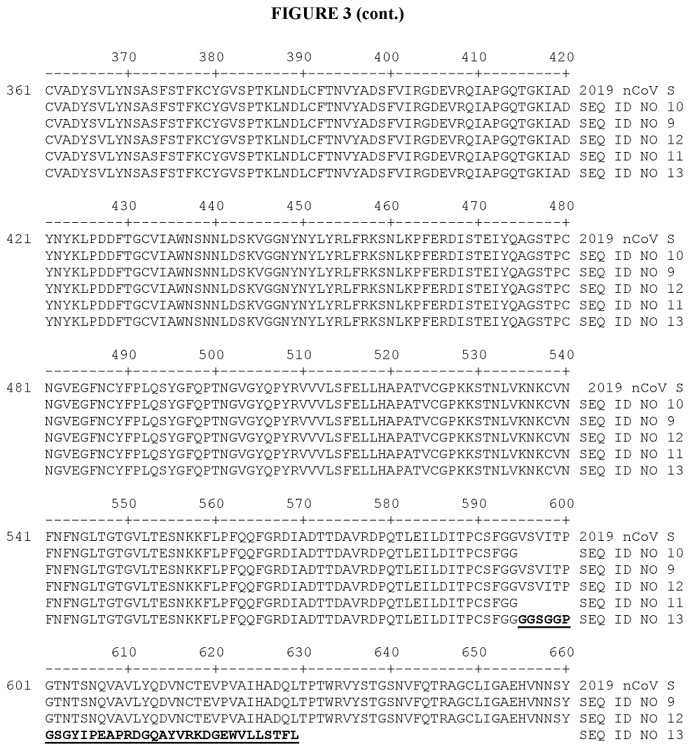

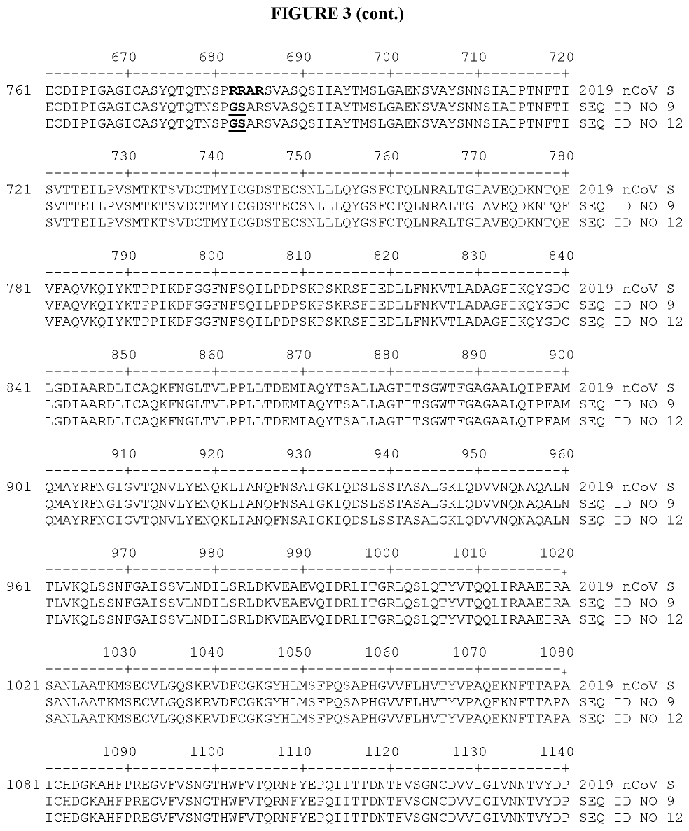

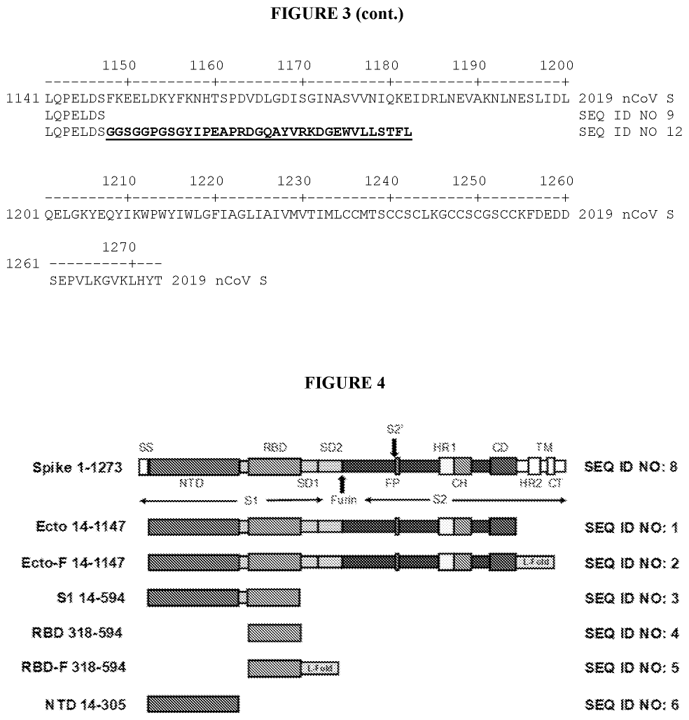

In a preferred embodiment of the invention, the SARS-CoV-2 spike subunit proteins have an N-terminus sequence with a synthetic sequence designed to enhance the processing of the secretion signal cleavage site by signal peptidase as the translated protein transits the endoplasmic reticulum. This synthetic cleavage site and N-terminal modification has been optimized to increase the recognition of the signal protease cleavage site. Specifically, the amino acid residues +1, +2, and +3 relative to the signal peptidase cleavage site have been modified. Upon cleavage, these amino acid residues define the N-terminus of the expressed SARS-CoV-2 protein subunit sequences. Following the cleavage by signal peptidase in S2 cells, the expressed SARS-CoV-2 spike protein subunits begin with the amino acid residues Ser, Ser, Asp. The N-terminal Ser, Ser, Asp (SSD) sequence can be seen in the SARS-CoV-2 spike subunit proteins shown in the amino acid alignment shown in FIG. 3.

In a preferred embodiment of the invention, the C-terminus of the SARS-CoV-2 spike ectodomain has been truncated to stabilize the soluble spike subunit protein that is expressed, SEQ ID NO: 9. Specifically, the C-terminus of the soluble SARS-CoV-2 spike ectodomain has been defined as Ser-1147 as shown in FIG. 3. The codon optimized nucleotide sequence that encodes the truncated C-terminal SARS-CoV-2 spike ectodomain is detailed in SEQ ID NO: 1.

In another aspect the codon optimized DNA sequence encodes the ectodomain, the optimized signal peptidase cleavage site, with a foldon domain at the C-terminal end to promote trimerization and includes SEQ ID NO: 2.

In another aspect, the codon optimized DNA sequence encodes the 51 subunit with the optimized signal peptidase cleavage site and includes SEQ ID NO: 3.

In a more preferred embodiment of the invention, the C-terminus of the SARS-CoV-2 spike 51 subunit has been truncated to stabilize the soluble spike subunit protein that is expressed, SEQ ID NO: 10. Specifically, the C-terminus of the soluble SARS-CoV-2 spike 51 subunit has been defined as Gly-594 as shown in FIG. 3. The codon optimized nucleotide sequence that encodes the truncated C-terminal SARS-CoV-2 spike S1 subunit is detailed in SEQ ID NO: 3.

In another aspect, the codon optimized DNA sequence encodes the RBD subunit with the optimized signal peptidase cleavage site and includes SEQ ID NO: 4.

In a more preferred embodiment of the invention, the SARS-CoV-2 spike RBD subunit has been truncated to stabilize the soluble spike subunit protein that is expressed, SEQ ID NO: 11. Specifically, the N-terminus and C-terminus of the soluble SARS-CoV-2 spike RBD subunit has been defined as Phe-318 and Gly-594, respectively, as shown in FIG. 3. The codon optimized nucleotide sequences that encode the truncated N- and C-terminal SARS-CoV-2 spike RBD subunits is detailed in SEQ ID NO: 4.

In another aspect the codon optimized DNA sequence encodes the RBD subunit, the optimized signal peptidase cleavage site, with a foldon domain at the C-terminal end to promote trimerization and includes SEQ ID NO: 5.

In a more preferred embodiment of the invention, the truncated the SARS-CoV-2 spike ectodomain and RBD subunits are further modified at the C-terminus by operably linking a foldon domain to the subunit to promote the trimerization of the expressed subunits, SEQ ID NO: 12 and SEQ ID NO: 13. The ectodomain-foldon and RBD-foldon relative to the ectodomain and RBD lacking the foldon domain are shown in FIG. 3. The codon optimized nucleotide sequences that encodes the SARS-CoV-2 spike ectodomain and RBD subunits with the linked foldon domains are detailed in SEQ ID NO: 2 and SEQ ID NO: 5.

Thus, the present invention provides the combination of multiple optimizations directed at different aspects of the SARS-CoV-2 spike gene sequence in such a manner that an additive benefit is achieved and results in high levels of the spike protein subunits being expressed. A summary of the SARS-CoV-2 spike subunit proteins expressed in Drosophila S2 cells relative to the full-length spike protein is presented in FIG. 4. The optimized SARS-CoV-2 spike sequence when used to express the defined protein subunits in Drosophila S2 cells results in the economic production of large quantities of high-quality proteins. The Examples below show that using the individual optimized elements in the SARS-CoV-2 gene sequence results in improved or enhanced expression of the spike protein subunits.

In another aspect, the codon optimized DNA sequence encodes the NTD subunit with the optimized signal peptidase cleavage site and includes SEQ ID NO: 6.

In another embodiment, the invention provides an isolated amino acid sequence encoded by a nucleic acid sequence selected from SEQ ID NO: 1, 2, 3, 4, 5 and 6.

The terms “peptide”, “polypeptide” and “protein” are used interchangeably herein and refer to any chain of at least two amino acids, linked by a covalent chemical bound. As used herein polypeptide can refer to the complete amino acid sequence coding for an entire protein or to a portion thereof. A “protein coding sequence” or a sequence that “encodes” a particular polypeptide or peptide, is a nucleic acid sequence that is transcribed (in the case of DNA) and is translated (in the case of mRNA) into a polypeptide in vitro or in vivo when placed under the control of appropriate regulatory sequences. The boundaries of the coding sequence are determined by a start codon at the 5′ (amino) terminus and a translation stop codon at the 3′ (carboxyl) terminus. A coding sequence can include, but is not limited to, cDNA from prokaryotic or eukaryotic mRNA, genomic DNA sequences from prokaryotic or eukaryotic DNA, and even synthetic DNA sequences. A transcription termination sequence will usually be located 3′ to the coding sequence.

In one aspect, the amino acid sequence includes SEQ ID NO: 9, 10, 11, 12, 13 or 14.

Polypeptides and polynucleotides that are about 85, 86, 87, 88, 89, 90, 91, 92, 93, 94, 95, 96, 97, 98, 99, 99.5% or more identical to polypeptides and polynucleotides described herein are embodied within the disclosure. For example, a polypeptide can have 80%, 85%, 90%, 91%, 92%, 93%, 94%, 95%, 96%, 97%, 98%, 99% or more identity to SEQ ID NOs:9-14.

Variants of the disclosed sequences also include peptides, or full-length protein, that contain substitutions, deletions, or insertions into the protein backbone, that would still leave at least about 70% homology to the original protein over the corresponding portion. A yet greater degree of departure from homology is allowed if like-amino acids, i.e. conservative amino acid substitutions, do not count as a change in the sequence. Examples of conservative substitutions involve amino acids that have the same or similar properties. Illustrative amino acid conservative substitutions include the changes of: alanine to serine; arginine to lysine; asparagine to glutamine or histidine; aspartate to glutamate; cysteine to serine; glutamine to asparagine; glutamate to aspartate; glycine to proline; histidine to asparagine or glutamine; isoleucine to leucine or valine; leucine to valine or isoleucine; lysine to arginine, glutamine, or glutamate; methionine to leucine or isoleucine; phenylalanine to tyrosine, leucine or methionine; serine to threonine; threonine to serine; tryptophan to tyrosine; tyrosine to tryptophan or phenylalanine; valine to isoleucine to leucine.

In another embodiment, the C-terminal end of the expressed RBD and 51 subunits is defined as Gly 594 to avoid the region of the spike protein with potential for immune enhancement. This modification is included in SEQ ID NO: 10, SEQ ID NO: 11, and SEQ ID NO: 13.

In an additional embodiment, the invention provides an expression vector including a nucleic acid sequence encoding a SARS-Cov-2 spike (S) protein, wherein the nucleic acid sequence includes SEQ ID NO: 1, 2, 3, 4, 5 or 6.

The term “synthetic” refers to sequences that are not found to occur naturally.

The term “operably linked” refers to a juxtaposition wherein the components so described are in a relationship permitting them to function in their intended manner.

“Expression cassette” means the combination of promoter elements with other transcriptional and translational regulatory control elements which are operably linked to a gene sequence to be expressed. A gene sequence can be inserted into the expression cassette for the purpose of expression of said gene sequence. The expression cassette is capable of directing transcription which results in the production of an mRNA for the desired gene product which is then translated to protein by the host cell translational systems. The expression cassette is integral to the expression vector (plasmid). Such an expression vector directs expression of the protein encoded by the gene sequence once introduced into host cells.

In one aspect, the vector is a Drosophila melanogaster expression vector.

The Drosophila melanogaster cell expression system (“Drosophila expression system”) is an established heterologous protein expression system based on the use of expression vectors containing Drosophila promoters and Drosophila S2 cells (“S2 cells”) (Schneider, Embryol. Exp. Morph. (1972) 27:353-365). S2 cells are transformed with these vectors in order to establish stable cell lines expressing proteins corresponding to the heterologous sequences introduced into the vector (Johansen, H. et al., Genes Dev. (1989) 3:882-889; Ivey-Hoyle, M., Curr. Opin. Biotechnol. (1991) 2:704-707; Culp, J. S., et al., Biotechnology (NY) (1991) 9:173-177; U.S. Pat. Nos. 5,550,043; 5,681,713; 5,705,359; 6,046,025). This insect cell expression system has been shown to successfully produce a number of proteins from different sources. Examples of proteins that have been successfully expressed in the Drosophila S2 cell system include HIV gp120 (Culp, J. S., et al., Biotechnology (NY) (1991) 9:173-177; Ivey-Hoyle, M., Curr. Opin. Biotechnol. (1991) 2:704-707), human dopamine β-hydrolase (Bin et al., Biochem. 1 (1996) 313:57-64), human vascular cell adhesion protein (Bernard et al., Cytotechnol. (1994) 15:139-144). In each of these examples, expression levels were greater than other expression systems that had been previously utilized.

In addition to high levels of expression, the Drosophila expression system has been shown to be able to express heterologous proteins that maintain native-like biological function (Bin et al., Biochem. 1 (1996) 313:57-64), (Incardona and Rosenberry, Mol. Biol. Cell. (1996) 7:595-611). More recent examples have shown by means of X-ray crystallography studies that this expression system is capable of producing molecules with native-like structure (Modis et al., Proc. Natl. Acad. Sci. USA (2003) 100:6986-6991), (Modis et al., Nature (2004) 427:313-319), (Xu et al., Acta. Crystallogr. D Biol. Crystallogr (2005) 61:942-950). Two other recent publications have also demonstrated the ability of the Drosophila expression system to produce high quality products. In the first report, Schmetzer et al. (J. Immun. (2005) 174: 942-952) compares baculovirus-expressed EpCAM protein to Drosophila-expressed EpCAM protein for protein folding and native conformation. Specifically, BES-expressed EpCAM and Drosophila-expressed EpCAM were compared to denatured Drosophila-expressed EpCAM. It was determined that the BES-expressed EpCAM was in a partial folded state relative to the non-denatured and denatured Drosophila-expressed EpCAM protein. This indicates that the BES-expressed protein is in an incompletely folded state. The Drosophila-expressed EpCAM protein, on the other hand, adopted a more completely folded state. The authors of this paper considered the Drosophila-expressed protein to be in the “natural” state while the baculovirus-expressed protein was not. In the second report, Gardsvoll et al. (Prot. Exp. Purif. (2004) 34:284-295) demonstrate that the expression of the urokinase-type plasminogen activator receptor (uPAR) in S2 cells results in a more homogeneous product in regard to glycosylation (5 N-linked sites) than uPAR expressed in CHO cells.

In some aspects, the expression cassette of the Drosophila expression vector pHH202 has a nucleic acid sequence including SEQ ID NO: 7.

In another aspect, the SARS-CoV-2 S protein has an amino acid sequence including SEQ ID NO: 9, 10, 11, 12, 13 or 14.

In another embodiment, the Drosophila expression vector pHH202 that includes SEQ ID NO: 1, SEQ ID NO: 2, SEQ ID NO: 3, SEQ ID NO: 4, SEQ ID NO: 5, or SEQ ID NO: 6 is used to express and secrete the encoded heterologous SARS-CoV-2 S protein subunits, SEQ ID NO: 9, SEQ ID NO: 10, SEQ ID NO: 11, SEQ ID NO: 12, SEQ ID NO: 13, or SEQ ID NO: 14, from cultured insect cells. The expression cassette of the pHH202 vector includes SEQ ID NO: 7.

In one embodiment, the expression vectors are used in Drosophila cells.

In another embodiment, the expression vectors are used in Drosophila S2 cells.

The term “transformed” refers to the DNA-mediated transformation of cells. This refers to the introduction of plasmid DNA into insect cells in the process of generating stable cell lines following the integration of the introduced DNA into the genome of the cells. This term is used in place of the term “transfection” which is often used in the same context. The term transformation is used for the introduction of plasmid DNA to cultured cells to distinguish from the introduction of viral DNA into cultured cells which was originally referred to as transfection. As there are no viral DNA sequences in the present invention which are introduced into the host cells that results in the production of virus-like particles or cell lysis the term transformed is preferred.

“Expression” or “expressed” means the production of proteins using expression vectors and host cells, for instance, Drosophila S2 cells to produce a recombinant protein product that is readily detectable as a cell-associated product or as a secreted product in the culture medium.

“Secretion” means secretion of an expressed recombinant protein from cultured host cells into culture medium. The expressed and secreted protein is the result of a given gene sequence being operably linked to an expression cassette such that the sequence codes for the given protein.

The term “product” refers to any recombinant protein, full length or subunit thereof, which is expressed by a host cell into which an expression vector carrying the gene sequence encoding the product has been introduced.

Insect cells are an alternative eukaryotic expression system that provide the ability to express properly folded and post-translationally modified proteins while providing simple and relatively inexpensive growth conditions. The use of stably transformed insect cell expression systems provide benefits over those based on baculovirus infection of the host insect cells. On this basis, S2 cells were selected as the insect host cells of choice. As a result, the efforts to optimize the expression vectors for stably transformed insect cells were based on data derived from the analysis of specific Drosophila genes as well as the complete Drosophila genome.

In one embodiment, the invention provides a method of producing a protein in vitro including an expression vector with an operably-linked nucleic acid sequence encoding a SARS-CoV-2 spike (S) protein, wherein the nucleic acid sequence includes SEQ ID NO: 1, 2, 3, 4, 5 or 6 with a Drosophila melanogaster cell and culturing the cell under conditions to produce the protein.

In one aspect, the Drosophila melanogaster cell is an Schneider 2 (S2) cell.

Vaccine Compositions

In another embodiment, the invention provides a vaccine composition including (a) an effective amount of a SARS-CoV-2 spike (S) subunit protein, wherein the S protein is encoded by a nucleic acid sequence including SEQ ID NO: 1, 2, 3, 4, 5 or 6, and (b) an effective amount of an adjuvant.

According to the invention, the term “vaccine” relates to a pharmaceutical preparation (pharmaceutical composition) or product that upon administration induces an immune response, in particular a cellular immune response, which recognizes and attacks a pathogen such as a virus, or a diseased cell such as an infected cell. A vaccine may be used for the prevention or treatment of a disease.

The terms “therapeutically effective amount”, “effective dose,” “therapeutically effective dose”, “effective amount,” “prophylactically effective amount”, “prophylactic dose,” “prophylactically effective dose”, “prophylactic amount,” or the like refer to that amount of the subject compound that will elicit the biological or medical response of a tissue, system, animal or human that is being sought by the researcher, veterinarian, medical doctor or other clinician. Generally, the response is either amelioration of symptoms in a patient or a desired biological outcome (e.g., induction of an immune response, prevention of viral infection, and the like). For example, a therapeutic or prophylactic dose of the vaccine composition described herein is a dose or amount of the vaccine composition that is sufficient to induce an immune response in the subject, the immune response being then sufficient to provide immune protection to the subject. The effective or prophylactic amount can be determined as described herein or using methods known to those of skill in the art.

The vaccine compositions described herein include an adjuvant. Vaccine adjuvants are critical for the effective development of protective responses with many antigens.

Toll-like receptor (TLR) agonist adjuvants are particularly promising, as they engage the innate immune system to stimulate a more robust and durable adaptive immune response. Ligands for TLR 7/8 (Imiquimod, Resiquimod), TLR-9 (CpG), TLR-5 (Flagellin), and TLR-4 have been evaluated pre-clinically as components of vaccine adjuvants. TLR-4 agonist adjuvants have been shown to be safe and effective in several clinical trials, and the TLR-4 agonist adjuvant MPL is a component of the licensed HPV vaccine Cervarix® (GlaxoSmithKline, Rixensart, Belgium).

TLR-4 agonist adjuvants can be combined with saponin adjuvants. The use of this combination of adjuvants results in potent and durable immune response.

An ideal TLR-4 agonist is a fully synthetic lipid A molecule (SLA). The most commonly used TLR-4 agonist used for vaccines is monophosphoryl lipid A (MPL). MPL is prepared from bacterial cell walls. The processes used result in heterogeneous preparations of MPL. The synthetic nature of SLA provides for more defined composition relative to MPL. Furthermore, the structure of SLA has been optimized to bind more effectively to the human TLR-4 receptor. SLA enhances the ability of the immune system to respond to vaccine antigens.

An ideal saponin adjuvant is a highly purified preparation derived from the Soap bark tree (Quillaja saponaria) and contains a water-soluble triterpene glucoside molecule. QS21 is a saponin-based adjuvant of this nature. QS21 is purified from extracts of the tree bark. QS21 enhances the ability of the immune system to respond to vaccine antigens. Another example of saponin derived adjuvant includes GPI-0100.

In one aspect, liposomes are combined with the SLA and QS21 adjuvants to form a liposome-based formulation. The liposome formulation containing SLA and QS21 is referred to as LSQ. The liposome composition can be either anionic or cationic nature, or more preferably it has a neutral charge. The liposome size range can vary from 20-300 nm, more preferably from 40-200 nm, and most preferably 50-150 nm in size.

In another aspect, a stable oil-in-water emulsion (SE) which preferably includes squalene is combined with SLA to form a stable oil-based emulsion.

In some aspects, the adjuvant is selected from the group consisting of GPI-0100, synthetic lipid A (SLA) in a stable oil-in water emulsion (SE) (SLA-SE), QS21, QS21 combined with SLA to form a liposome formulation (SLA-LSQ), and QS21+CpG.

The vaccine compositions described herein include a S subunit protein that is recombinantly produced and expressed in insect host cells. The host cells are modified (e.g., transformed) to express any one of the expression vectors including the nucleic acid sequences described herein, encoding a S protein (e.g., SEQ ID NO: 1, 2, 3, 4, 5 or 6). The resulting proteins, recombinantly produced by the host cells are used in the formulation of the vaccine compositions described herein.

In one aspect, the S protein of the vaccine composition is the 51 subunit protein. In some aspects, the S1 subunit protein is encoded by a nucleic acid sequence comprising SEQ ID NO: 3. In another aspect, the S protein is recombinantly produced and expressed in insect host cells.

In one aspect, the S protein of the vaccine composition is the S1 subunit protein encoded by a nucleic acid sequence comprising SEQ ID NO: 3 and the adjuvant of the vaccine composition is SLA-SE.

In some aspects, the vaccine composition further includes a pharmaceutically acceptable excipient or carrier.

The vaccine formulation of the present invention may further include one or more additional pharmaceutically acceptable diluents, carriers, solubilizers, emulsifiers, preservatives and/or adjuvants. By “pharmaceutically acceptable” it is meant the carrier, diluent, solubilizer, emulsifier, preservative or excipient must be compatible with the other ingredients of the formulation and not deleterious to the recipient thereof. For example, a pharmaceutical composition may contain formulation materials for modifying, maintaining or preserving, for example, the pH, osmolarity, viscosity, clarity, color, isotonicity, odor, sterility, stability, rate of dissolution or release, adsorption or penetration of the composition. Suitable formulation materials include, but are not limited to, amino acids (such as glycine, glutamine, asparagine, arginine or lysine); antimicrobials; antioxidants (such as ascorbic acid, sodium sulfite or sodium hydrogen-sulfite); buffers (such as borate, bicarbonate, Tris-HCl, citrates, phosphates or other organic acids); bulking agents (such as mannitol or glycine); chelating agents (such as ethylenediamine tetraacetic acid (EDTA)); complexing agents (such as caffeine, polyvinylpyrrolidone, beta-cyclodextrin or hydroxypropyl-beta-cyclodextrin); fillers; monosaccharides; disaccharides; and other carbohydrates (such as glucose, mannose or dextrins); proteins (such as serum albumin, gelatin or immunoglobulins); coloring, flavoring and diluting agents; emulsifying agents; hydrophilic polymers (such as polyvinylpyrrolidone); low molecular weight polypeptides; salt-forming counterions (such as sodium); preservatives (such as benzalkonium chloride, benzoic acid, salicylic acid, thimerosal, phenethyl alcohol, methylparaben, propylparaben, chlorhexidine, sorbic acid or hydrogen peroxide); solvents (such as glycerin, propylene glycol or polyethylene glycol); sugar alcohols (such as mannitol or sorbitol); suspending agents; surfactants or wetting agents (such as pluronics, PEG, sorbitan esters, polysorbates such as polysorbate 20, polysorbate 80, triton, tromethamine, lecithin, cholesterol, tyloxapal); stability enhancing agents (such as sucrose or sorbitol); tonicity enhancing agents (such as alkali metal halides, preferably sodium or potassium chloride, mannitol sorbitol); delivery vehicles; diluents; excipients and/or pharmaceutical adjuvants. (Remington's Pharmaceutical Sciences, 18th Edition, A. R. Gennaro, ed., Mack Publishing Company (1990).

Methods of Use

In one embodiment, the invention provides a method of preventing a SARS-CoV-2 entry into a cell including contacting the cell with a therapeutically or prophylactically effective amount of composition including (a) an effective amount of a SARS-CoV-2 spike (S) protein, wherein S protein is encoded by a nucleic acid sequence including SEQ ID NO: 1, 2, 3, 4, 5 or 6, and (b) an effective amount of an adjuvant, thereby preventing SARS-CoV-2 entry into the cell.

SARS-CoV-2 is capable of infecting cells by interacting with human angiotensin converting enzyme 2 (hACE2), which acts as a receptor for the virus S protein. By “preventing entry” of the virus into a cell, it is meant that the immune response elicited by the vaccine composition described herein prevents the interaction of the virus S protein with its receptor at the surface of the cell. Thus, the interaction can be reduced, inhibited or prevented by administration of the vaccine composition described herein.

In one aspect, the vaccine induces the production of nAbs in the subject.

By “neutralizing antibody” or “nAb” it is meant that the vaccine induces the production of an antibody that defends a cell from a pathogen or infectious particle by neutralizing any effect it has biologically. Neutralization renders the particle, such as the virus, no longer infectious or pathogenic. Neutralizing antibodies are part of the humoral response of the adaptive immune system against viruses, intracellular bacteria and microbial toxin. By binding specifically to surface structures (such as the spike protein of SARS-CoV-2) on an infectious particle, nAbs prevent the particle from interacting with its host cells and thus, prevent entry of the of the virus into the host cell. If the immune response, nAbs, are able to eliminate the infectious particles before any infection takes place, this is known as sterilizing immunity.

In some aspects, the nAbs prevent the binding of a SARS-CoV-2 to a target cell and/or target receptor. In various aspects, the target receptor is an ACE2 receptor.

In another embodiment, the invention provides a method of stimulating a protective immune response in a subject including administering to the subject a therapeutically or prophylactically effective amount of composition including (a) an effective amount of a SARS-CoV-2 spike (S) protein wherein S protein is encoded by a nucleic acid sequence including SEQ ID NO: 1, 2, 3, 4, 5 or 6, and (b) an effective amount of an adjuvant, thereby stimulating a protective immune response.

The term “subject” as used herein refers to any individual or patient to which the subject methods are performed. Generally, the subject is human, although as will be appreciated by those in the art, the subject may be an animal. Thus other animals, including vertebrate such as rodents (including mice, rats, hamsters and guinea pigs), cats, dogs, rabbits, farm animals including cows, horses, goats, sheep, pigs, chickens, etc., and primates (including monkeys, chimpanzees, orangutans and gorillas) are included within the definition of subject.

The terms “administration of” and or “administering” should be understood to mean providing a pharmaceutical composition in a therapeutically or prophylactically effective amount to the subject in need of treatment. Administration routes can be enteral, topical or parenteral. As such, administration routes, in general, include but are not limited to intracutaneous, subcutaneous, intravenous, intraperitoneal, intraarterial, intrathecal, intracapsular, intraorbital, intracardiac, intradermal, transdermal, transtracheal, subcuticular, intraarticulare, subcapsular, subarachnoid, intraspinal and intrasternal, oral, sublingual, buccal, rectal, vaginal, nasal ocular administrations, as well infusion, inhalation, and nebulization. The phrases “parenteral administration” and “administered parenterally” as used herein means modes of administration other than enteral and topical administration.

To immunize subjects against SARS-CoV-2-induced disease for example, the vaccine formulation containing the recombinant subunit protein and adjuvant are administered to the subject in conventional immunization protocols involving, usually, multiple administrations of the vaccine. Administration is typically by injection, typically intramuscular or subcutaneous injection; however, other systemic modes of administration may also be employed.

In one aspect, administering includes injecting two doses to the subject at a 3-weeks interval.

Many different techniques exist for the timing of the immunizations when a multiple administration regimen is utilized. Generally, it is preferable to use a vaccine more than once to increase the levels and diversities of expression of the immunoglobulin repertoire expressed by the immunized subject. Typically, if multiple immunizations are given, they will be given one to two months apart. To further boost the immune response, a second dose of vaccine can be administered. The preferred immunization schedule for two doses is 0 and 3 weeks. Other immunizations schedules can also be utilized. For example, alternative immunization schedules such as 0, 1; 0, 2 or 0, 3 months could be used. Additional booster vaccinations may be administered at prescribed intervals such as every 5 to 10 years.

In another aspect, administering includes injecting intramuscularly. In some aspects, a dose includes about 0.5-50 μg of purified S protein. For example, a dose includes about 0.5, 1, 2, 5, 10, 15, 20, 25, 30, 35, 40, 45, or 50 μg of purified S protein.

By “stimulating, inducing, or eliciting a protective immune response”, it is meant that the vaccine composition described herein induces an immune response in the subject, which provides the subject with an immune protection against a disease. “Inducing an immune response” may mean that there was no immune response against a particular antigen before induction, but it may also mean that there was a certain level of immune response against a particular antigen before induction and after induction said immune response is enhanced. Thus, “inducing an immune response” also includes “enhancing an immune response”. Preferably, after inducing an immune response in a subject, said subject is protected from developing a disease such as a COVID-19 disease or the disease condition is ameliorated by inducing an immune response.

The term “immune response” refers to an integrated bodily response to an antigen and preferably refers to a cellular immune response and or a humoral immune response. The immune response may be protective/preventive/prophylactic.

In one aspect, the immune response is a balanced immune response.

Immune response includes cellular and humoral immune responses. The humoral immune response is mediated by antibodies secreted by B cells. The antibodies neutralize and opsonize free extracellular pathogens, and prolonged antibody production lasting for years after infection or vaccination provide the first line of defense by the adaptive immune system. The cellular immune response is mediated by antigen specific CD4+ and CD8+ T cells and cells of the innate immune system (e.g. dendritic cells, NK cells and macrophages). T cells cannot recognize free pathogens, but instead identify infected cells and exert effector functions including direct cytotoxic effect and cytokine release. CD4+ T cells can be divided into two major subsets, type 1 helper cells (Th1) that secrete interleukin-2 and interferon-γ, and type 2 helper T cells (Th2) secreting interleukins-4, 5, 6 and 10. The cytokines produced by Th1 cells promote a cell-mediated immune response, whereas the humoral immune response is triggered and maintained by cytokines secreted by Th2 cells following antigenic exposure. The IgG antibody subclass distribution elicited after vaccination is also indicative of the type of immune response, as the IgG1 subclass in mice is believed to signal a Th2 response whilst the IgG2a subclass indicates more of a Th1 profile.

A “balanced immune response” is characterized by an immune response that include both a strong humoral and a strong cellular immune response, as can be measured by evaluating a ratio between Th1 and Th2 profiles, such as by establishing a IgG2a:IgG1 ratio.

In one aspect, a balanced immune response is characterized by a IgG2a:IgG1 ratio that is equal or greater than 1.

In an additional embodiment, the invention provides a method of inhibiting a SARS-CoV-2 infection in a subject including administering to the subject a therapeutically or prophylactically effective amount of composition including (a) an effective amount of a SARS-CoV-2 spike (S) protein, wherein S protein is encoded by a nucleic acid sequence including SEQ ID NO: 1, 2, 3, 4, 5 or 6, and (b) an effective amount of an adjuvant, thereby inhibiting SARS-CoV-2 infection.

In another embodiment the invention provides a method of inhibiting transmission of a SARS-CoV-2 infection by a subject including administering to the subject a therapeutically or prophylactically effective amount of composition including (a) an effective amount of a SARS-CoV-2 spike (S) protein, wherein S protein is encoded by a nucleic acid sequence including SEQ ID NO: 1, 2, 3, 4, 5 or 6, and (b) an effective amount of an adjuvant, thereby inhibiting transmission of a SARS-CoV-2 infection.

By “inhibiting infection”, it is meant that the immune response elicited by the vaccine composition prevents the entry of the virus into cells in the subject, thereby blocking the replication and infection cycle of the virus. As previously discussed, the induction of the production of nAbs by the vaccine prevents the binding of SARS-CoV-2 to its target cell and/or target receptor, and thus prevents the infection of the target cells by the virus.

By “inhibiting transmission” it is meant that the vaccine composition, by preventing the entry of the virus into cells of a subject inhibits the replication and infection cycle of the virus, and therefore reduces or inhibits the risk of transmitting the virus to another subject, by reducing the viral load in the vaccinated subject.

In many aspects, the vaccine induces the production of nAbs in the subject. In some aspects, the nAbs prevent the binding of a SARS-CoV-2 to a target cell and/or target receptor. In various aspects, the target receptor is an ACE2 receptor.

In one aspect, the adjuvant is selected from the group consisting of GPI-0100, QS21+CpG, SLA-SE, and SLA-LSQ.

In other aspects, administering the vaccine to the subject increases the subject survival.

The vaccine composition described herein induces the production of nAbs in the subject and prevents SARS-CoV-2 entry into cells of the subject, thereby inhibiting SARS-CoV-2 infection and transmission in/by the subject. COVID 19 disease, the respiratory disease induced by SARS-CoV-2 infection is a deadly disease. By inhibiting SARS-CoV-2 infection and transmission in/by the subject, the vaccine composition described herein prevents the development of SARS-CoV-2-related disease COVID-19, and therefore increase the survival of the subject.

In some aspects, administering the vaccine prevents the development of a COVID-19 disease in the subject.

Although the descriptions presented above and the examples that follow are primarily directed at the use of the optimized expression vectors with Drosophila S2 cells, the vectors and methods can be applied to other insect cell lines that result in stable cell lines following transformation of host cells with plasmid DNA.

Presented below are examples discussing expression of SARS-CoV-2 Spike protein subunits, the optimized polynucleotides encoding them, and expression vector thereof contemplated for use in the discussed applications. The following examples are provided to further illustrate the embodiments of the present invention but are not intended to limit the scope of the invention. While they are typical of those that might be used, other procedures, methodologies, or techniques known to those skilled in the art may alternatively be used.

The following examples are offered by way of illustration and not by way of limitation.

### EXAMPLES

The following examples describe the development of the optimized SARS-CoV-2 gene sequences for the expression of the envelope protein in insect cells. The examples demonstrate the ability to effectively express the proteins in Drosophila S2 cells at levels that are commercially suitable for product development.

The examples demonstrate the ability to enhance expression of SARS-CoV-2 proteins in S2 cells and the efforts made to determine the contribution of various changes to enhance the expression and function of the spike protein subunits. The results presented below demonstrate that different modifications and truncations of the spike protein can result in high levels of expression or in products that are not properly folded or glycosylated. Thus, the selection of effective modifications must be determined thorough experimentation. Hence, the invention described herein is unique in that the modifications and truncations described result in distinct protein subunits suitable for use as vaccines to protect against disease caused by infection with SARS-CoV-2.

### Example 1

### Expression of Wild Type and Codon Optimized SARS-Cov-2 Spike Ectodomain in Drosophila S2 Cells

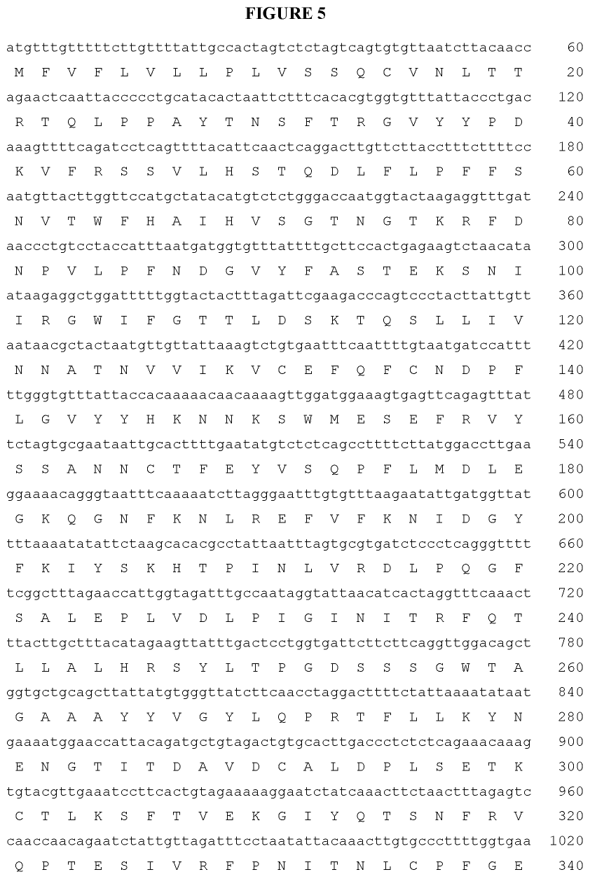

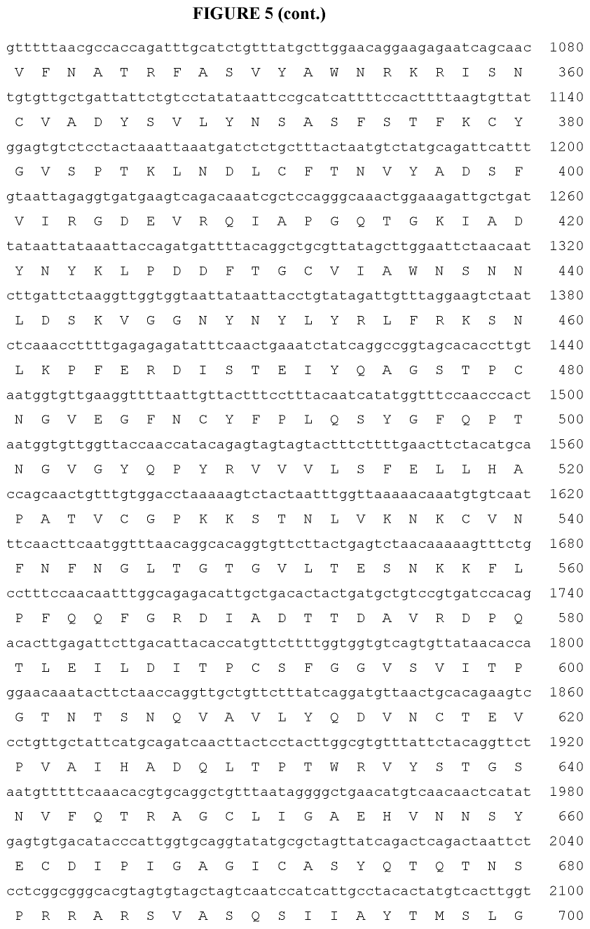

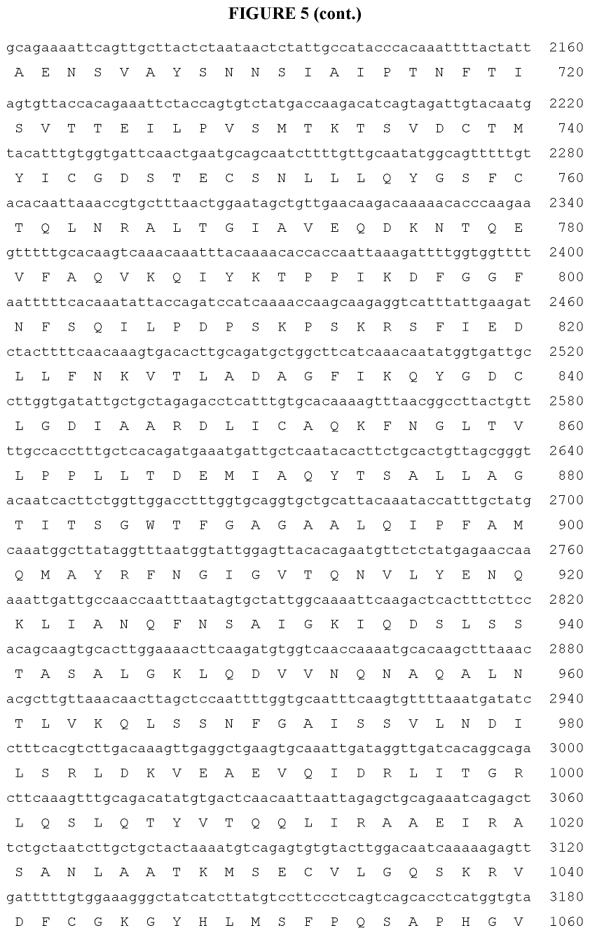

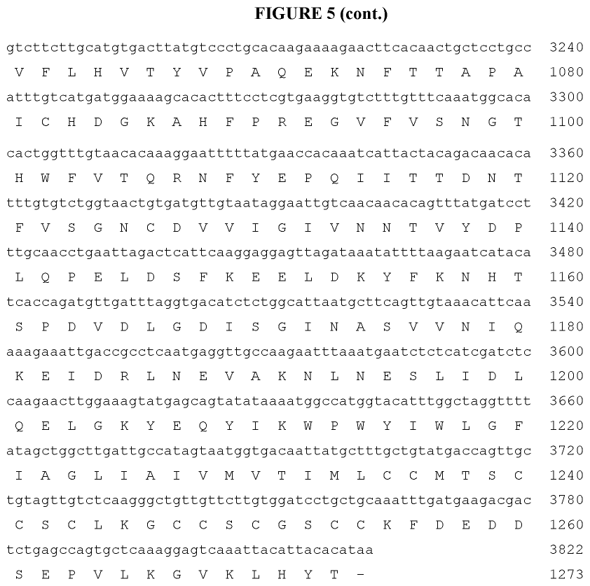

In an effort to identify optimized gene sequences for driving high levels of high-quality SARS-CoV-2 spike protein in S2 cells, the wild type spike gene ectodomain (ecto) sequence (S-WT-Ecto) was compared to a codon optimized spike gene ectodomain sequence (S-Ecto-CO). Both the wild type and codon optimized sequences were produced synthetically (ATUM, Santa Clara, CA). For codon optimization, the standard Drosophila melanogaster codon table was used (Kazusa DNA Research Institute, www.kazusa.or.jp/codon/). As the objective is to improve the efficiency of expression, which is in part controlled by the translation process, a threshold of 10% usage was used in assigning codons (any codon that is used <10% is excluded). Additionally, based on our analysis of highly expressed proteins in Drosophila, we have added the exclusion of the following codons, CGA for Arg, ATA for Ile, and GTA for Val. The synthesized gene sequences included appropriate restriction enzyme sites at the ends and a stop codon was included at the end of the envelope protein coding region. For the expression of the SARS-CoV-2 spike protein subunits, the genomic sequence representing the SARS-CoV-2 spike protein was used. The sequences utilized for expression are based on the SARS-CoV-2 Wuhan-Hu-1 strain (Genbank Accession number NC 045512). The wild type Wuhan-Hu-1 spike nucleotide sequence (SEQ ID NO: 15) along with translation (SEQ ID NO: 8) is provided in FIG. 5. The sequence for spike protein was truncated at the carboxy end at Ser-1147 to produce the ectodomain (S-Ecto). Additionally, the native furin cleavage site RRAR (682-685) that defines the S1/S2 junction was mutated to remove the cleavage site and further stabilize the expressed ectodomain. The sequence was mutated to encode GSAR.

To improve the processing at the signal peptide cleavage site, the three amino acids, Ser-Ser-Asp, were added immediately before the N-terminal Gln (Q) residue that results from the following cleavage of the signal peptide. The sequence that encodes Ser-Ser-Asp (SSD) was fused in frame with the secretion signal of the expression vector. When the S-SSD-Ecto sequence is expressed in the S2 cells, the S-Ecto junction is processed by an S2 encoded signal peptidase. This results in the secretion of an S-Ecto product into the culture medium with an N-terminus of Ser-Ser-Asp-Gln (SSDQ). The codon optimized nCoV-S-SSDQ-Ecto-CO sequence with the enhanced N-terminus and the mutated furin site is detailed in SEQ ID NO: 1. The resultant protein product is detailed in SEQ ID NO: 9. The alignment of the nCoV S SSDQ Ecto CO protein product relative to the wild type SARS-CoV-2 S protein is shown in FIG. 3.

As a control, the codon optimized S-Ecto was also fused to the secretion signal of the expression vector using the native N-terminus (Gln) lacking the SSD sequence. This is referred to as nCoV-S-SSD-SQ-Ecto-CO.

The synthetic DNA fragments were digested with appropriate restriction enzymes and inserted within the expression cassette (SEQ ID NO: 7) of the pHH202 expression vector that has been digested with Nhe I and Xho I. The pHH202 expression cassette contains the following elements: metallothionein promoter, optimized Kozak sequence, influenza HA secretion signal, and the SV40 early 3′UTR. The hygromycin encoding gene is also incorporated into the pHH202 expression plasmid downstream of the expression cassette. The pHH202 expression plasmid is designed to allow directional cloning of the gene of interest into unique Nhe I and Xho I sites. The junctions and full inserts of all constructs were sequenced to verify that the various components that have been introduced are correct and that the proper reading frame has been maintained.

Standard methods of culturing and transformation of S2 cells were utilized (Van der Straten, Methods in Mol. and Cell Biol. (1989) 1:1-8; Culp et al., Biotechnology (1991) 9:173-177; Kirkpatrick and Shatzman, In Gene Expression Systems: Using Nature for the Art of Expression, Eds. Fernandez and Hoeffler, Academic Press, (1999) 289-330). Drosophila S2 cells (Schneider, J. Embryol. Exp. Morph. (1972) 27:353-365) obtained from ATCC were utilized. The S2 cells have been adapted to growth in Excell 420 medium (Sigma, St Louis, MO) and all procedures and culturing described herein were in Excell 420 medium. Cultures are typically seeded at a density of 2×10⁶ cells/mL and are passed between days 5 and 7. All cultures were incubated at 26° to 27° C. Expression plasmids into which genes of interest were inserted were transformed into S2 cells using the ExpiFectamine Sf reagent (ThermoFisher, Waltham, MA). Following transformation, cells resistant to hygromycin B, 0.3 mg/mL, were selected. Once stable cell lines were selected, they were evaluated for expression of the appropriate products. For the evaluation of expression, 2 mL cultures of selected cell lines were seeded at 2×10⁶ cells/mL and cultured in the presence of 0.2 mM copper sulfate at 26° C. for 6 days. Cultures were evaluated for expression of recombinant proteins in both the cell associated fractions and the culture medium. Proteins were separated by SDS-PAGE and either stained with Coomassie blue or transferred onto nitrocellulose for Western blot analysis. Expression levels ≥5 μg/mL (5 mg/L) are readily detected in S2 cultures by Coomassie staining of SDS-PAGE gels.

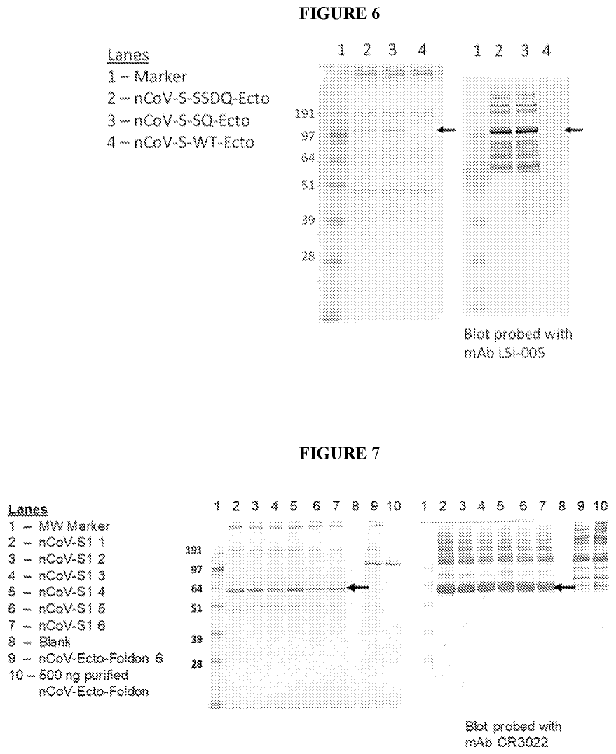

Parental S2 cell lines expressing the nCoV-S-SSDQ-Ecto-CO, nCoV-S-SQ-Ecto-CO and nCoV-S-WT-Ecto, were established as described above. The expression data for the parental S2 cell lines expressing two codon-optimized S-Ecto constructs and the WT S-Ecto is shown in FIG. 6. Purified nCoV-S-Ecto-Foldon is included for comparison. The expression of the nCoV-S-Ecto products has been confirmed using the conformationally sensitive monoclonal antibody (mAb) CR3022 that recognizes most SARS coronavirus spike proteins. The mAb CR3022 is also used for purification utilizing immunoaffinity chromatography (IAC) methods. The mAb CR3022 based IAC purification methods are based on processes that have been successfully transferred to cGMP manufacturing for other protein subunits produced in S2 cells.

The use of the codon optimized nCoV-S-SSDQ-Ecto-CO or nCoV-S-SQ-Ecto-CO gene sequence in transformed S2 cells resulted in the expression nCoV-S-Ecto at approximately 5 mg/L, whereas the nCoV-S-WT-Ecto failed to result in detectable expression of the S-Ecto protein in the S2 cells. The lack of expression from the nCoV-S-WT-Ecto and the expression from the two codon optimized sequences demonstrates the benefits of the optimizations to drive improved expression.

### Example 2

### Expression of Codon Optimized SARS-Cov-2 Spike S1 Subunit in Drosophila S2 Cells

The SARS-CoV-2 S protein is divided into the S1 and S2 domains as shown in FIG. 1. The S1/S2 junction is delineated by a furin protease cleavage site. In an effort to further truncate the S protein a S1 subunit was designed to further focus the immune response. However, to further enhance the subunit as a vaccine candidate by avoiding sequences that could distract from the most desired epitopes, those that induce nAbs, the S1 subunit was truncated at Gly-594 at the C-terminal end. This is in contrast to truncating the S1 at Pro-681 that immediately precedes the furin cleavage site that defines the S1/S2 junction. The truncation at Gly-594 avoids a region around Asp-614 that has been implicated as a B-cell epitope that induces antibodies that enhance infection in SARS-CoV (Wang et al, 2016). This same location in SARS-CoV-2 has been identified to contain a mutation, Asp 614 to Gly, that results in higher virus titers, but also may have an immunological role (Korber et al, 2020). As with the codon optimized nCoV-S-Ecto subunit, the N-terminus of the S1 subunit is preceded with the SSD amino acids to ensure optimal cleavage during secretion. The defined S1 subunit relative to the full-length S protein can be seen in FIG. 3.

The codon optimized nucleotide sequence that encodes the C-terminal truncated SARS-CoV-2-S-S1 subunit is detailed in SEQ ID NO: 3 and the expressed and stabilized soluble S1 subunit protein is detailed in SEQ ID NO: 10.

Parental S2 cell lines expressing the nCoV-S-S1-CO have been established as described. The expression data for six parental S2 cell lines expressing codon optimized nCoV-S-S1 is shown in FIG. 7. Purified nCoV-S-Ecto-Foldon is included for comparison. The expression of the nCoV-S-S1 product has been confirmed using the conformationally sensitive mAb CR3022. The nCoV-S-S1 protein subunit can also be purified using the mAb CR3022 based IAC methods.

The use of the codon optimized nCoV-S-S1-CO gene sequence in transformed S2 cells resulted in the expression nCoV-S-S1 at >50 mg/L. This expression level is approximately 10-fold greater than the expression of the codon optimized nCoV-S-Ecto-CO gene sequence.

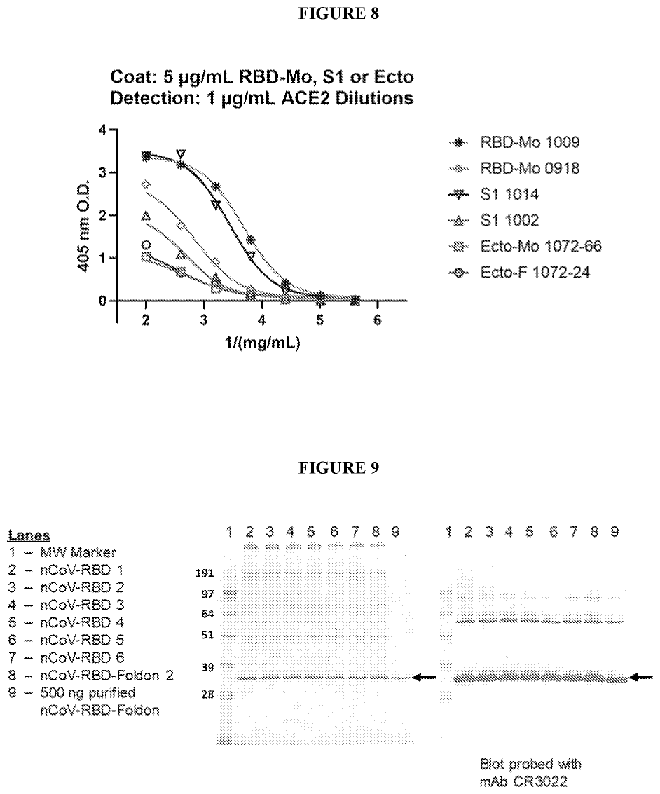

The functionality of nCoV-S-S1 subunit was confirmed in a binding assay with the recombinant hACE2 receptor. The nCoV-S-S1 subunit is bound to the ELISA plate and His-tagged recombinant hACE2 protein is then titered to determine the binding potential of the hACE2 to the expressed S1 subunit. The bound hACE2 is detected by alkaline phosphatase conjugated mouse anti-His. The results of the assay are shown in FIG. 8. From this data the functionality of the nCoV-S-S1 in terms of its ability to bind to the hACE2 protein has been confirmed.

### Example 3

### Expression of Codon Optimized SARS-Cov-2 Spike RBD Subunit in Drosophila S2 Cells

While a main goal is to optimize the expression of the SARS-CoV-2 spike protein as described in Examples 1 and 2, an equally important goal is to define spike protein subunits that can focus the immune response to critical epitopes in terms of defining potential vaccine candidates. For the SARS-CoV-2 spike protein, epitopes eliciting nAb responses are desired. The receptor binding domain (RBD) is a primary target as this domain plays a critical role in viral attachment to cells through the ACE2 receptor. Antibodies that can block the RBD/ACE2 interaction have the potential to block virus infection and thus prevent disease. Therefore, targeting a spike protein RBD subunit is an obvious choice for a vaccine candidate. While the RBD domain has been defined in the context of the full SARS-CoV-2 spike protein, the boundaries of the RBD in terms of the ability to express the domain in various cell-based expression systems is not well-defined.

In order to stabilize the SARS-CoV-2 spike RBD subunit, the N- and C-terminal ends have been defined to ensure proper folding. Specifically, the N-terminus and C-terminus of the soluble SARS-CoV-2 spike RBD subunit has been defined as Phe-318 and Gly-594, respectively, as shown in FIG. 3. The selection of this segment of the spike protein ensure that 8 cysteine residues are included that form 4 disulfide bonds and that N- and C-terminal lengths are present for stabilization of the RBD domain. As with the optimized nCoV-S-Ecto subunit, the N-terminus of the RBD subunit is preceded with the SSD amino acids to ensure optimal cleavage during secretion. The codon optimized nucleotide sequence that encodes the truncated N- and C-terminal SARS-CoV-2 spike RBD subunit is detailed in SEQ ID NO: 4 and the expressed and stabilized soluble spike subunit protein is detailed in SEQ ID NO: 11.

Parental S2 cell lines expressing the nCoV-S-RBD-CO have been established as described. The expression of the nCoV-S-RBD product has been confirmed using the conformationally sensitive mAb CR3022. The expression data for two parental S2 cell lines expressing codon optimized nCoV-S-RBD is shown in FIG. 9. Purified nCoV-S-RBD-Foldon is included for comparison. The expression of the nCoV-S-S1 product has been confirmed using the conformationally sensitive mAb CR3022. The nCoV-S-RBD protein subunit can also be purified using the mAb CR3022 based IAC methods.

The use of the codon optimized nCoV-S-RBD-CO gene sequence in transformed S2 cells resulted in the expression of nCoV-S-RBD at approximately 100 mg/L. This expression level is approximately 2-fold greater than the expression of the codon optimized nCoV-S-S1-CO gene sequence and approximately 20-fold greater than the expression of the codon optimized nCoV-S-Ecto-CO gene sequence.

The functionality of nCoV-S-RBD subunit was confirmed in a binding assay with the recombinant hACE2 receptor. The nCoV-S-RBD subunit is bound to the ELISA plate and His-tagged recombinant hACE2 protein is then titered to determine the binding potential of the hACE2 to the expressed RBD subunit. The bound hACE2 is detected by alkaline phosphatase conjugated mouse anti-His. The results of the assay are shown in FIG. 8. From this data the functionality of the nCoV-S-RBD in terms of its ability to bind to the hACE2 protein has been confirmed.

### Example 4

### Expression of Codon Optimized SARS-Cov-2 Spike Ectodomain and RBD Subunits with Foldon Domains in Drosophila S2 Cells

The surface proteins of enveloped viruses form multimeric configurations. The spike protein on the surface of coronaviruses is defined as a class 1 fusion protein as it forms a homotrimer. These trimeric structures are anchored in the virus membrane shell by the transmembrane (TM) domains. Expression of the spike protein ectodomain (lacking the TM) results in monomers. Various methods are available to drive the formation of multimeric forms of expressed proteins. In general, these methods involve the fusion of protein domains to the protein of interest. These protein domains can promote the formation of multimeric states of the protein. A commonly used protein domain used to promote trimer formation consist of a 29 amino acid sequence that is derived from the bacteriophage T4 fibritin protein sequence. This is referred to as the “foldon” sequence. The foldon sequence is located at the C-terminus of the fibritin protein, and naturally brings together three monomers of fibritin via non-covalent bonding to form a trimeric molecule. The use of the foldon domain provides a means to drive the formation of trimeric molecules when fused to the protein of interest.

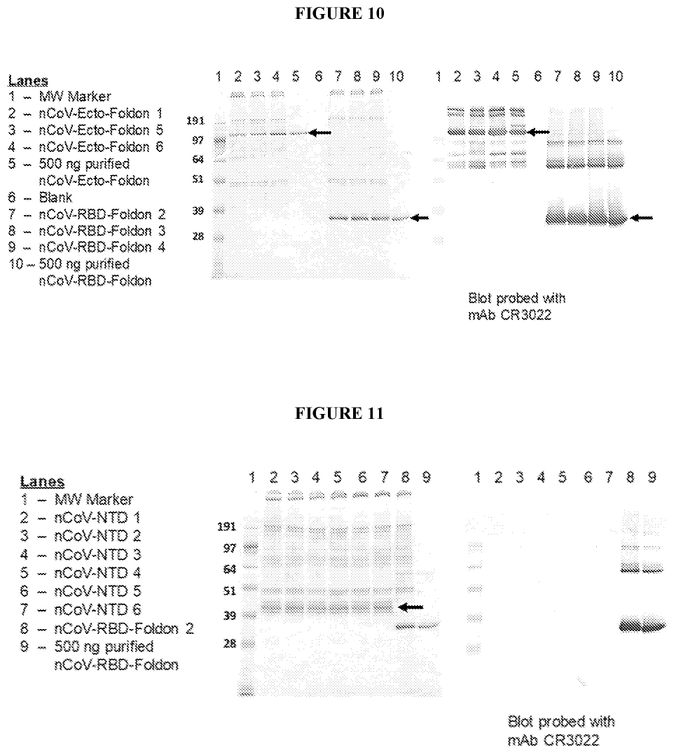

The truncated SARS-CoV-2 spike ectodomain and RBD subunits are further modified at the C-terminus by operably linking a foldon domain to the subunit to promote the trimerization of the expressed subunits, SEQ ID NO: 12 and SEQ ID NO: 13. The expression of the nCoV-S-Ecto-foldon and nCoV-S RBD-foldon are shown in FIG. 10. The expression of the nCoV-S-Ecto-foldon and nCoV-S RBD-foldon products has been confirmed using the conformationally sensitive mAb CR3022. The nCoV-S-Ecto-foldon and nCoV-S RBD-foldon protein subunits can also be purified using the mAb CR3022 based IAC methods.

The codon optimized nucleotide sequences that encodes the SARS-CoV-2 spike ectodomain and RBD subunits with the linked foldon domains are detailed in SEQ ID NO: 2 and SEQ ID NO: 5 and the expressed spike subunit protein with the C-terminal foldon domains are detailed in SEQ ID NO: 12 (Ecto-foldon) SEQ ID NO: 11 (RBD-Foldon).

The use of the codon optimized nCoV-S-Ecto-foldon-CO and nCoV-S-RBD-foldon-CO gene sequences in transformed S2 cells resulted in the expression of nCoV-S-Ecto-foldon-CO and nCoV-S-RBD-foldon-CO at approximately 50 mg/mL and 100 mg/L, respectively.

### Example 5

### Expression of Codon Optimized SARS-Cov-2 Spike NTD in Drosophila S2 Cells

As indicated in Example 3, an important goal is to define spike protein subunits that can focus the immune response to critical epitopes in terms of defining potential vaccine candidates. For the SARS-CoV-2 spike protein, epitopes eliciting nAb responses are desired. While the RBD is a primary target for nAb's, the N-terminal domain (NTD) is also a potential site for nAb epitopes. Therefore, targeting a spike protein NTD subunit is another choice for a potential vaccine candidate.

The NTD subunit was truncated at Ser-305 for the C-terminal end. As with the optimized nCoV-S-Ecto subunit, the N-terminus of the 51 subunit is preceded with the SSD amino acids to ensure optimal cleavage during secretion. The defined NTD subunit relative to the RBB-Foldon protein is shown in FIG. 11. The NTD subunit was not recognized by mAb CR3022. The codon optimized nucleotide sequence that encodes the SARS-CoV-2-S-NTD subunit is detailed in SEQ ID NO: 6 and the expressed and stabilize the soluble NTD subunit protein is detailed in SEQ ID NO: 14.

Initial attempts to express the spike protein subunit NTD resulted in multiple forms of the NTD that were misfolded and were heterogenous in the extent of glycosylation. Expression level in the S2 cells was approximately 20 mg/L.

### Example 6

### Immunogenic Evaluation of Codon Optimized SARS-Cov-2 Spike RBD-Foldon Proteins in Mice

The immunogenicity of the Drosophila S2 expressed codon optimized nCoV-S-RBD-Foldon subunit was evaluated in both Balb/c mice with 3 different adjuvants to assess immunogenic potential. Mice were immunized intra-muscularly with either two or 3 doses of nCoV-S-RBD-Foldon separated by 3 weeks intervals. A quantity of 5.0 μg of nCoV-S-RBD-Foldon was used for all doses. The 3 adjuvants tested were Alhydrogel, GPI-0100, and SLA-LSQ. Mice were bled 2 weeks after the 2^{nd} or 3^{rd} dose to prepare serum samples for antibody analysis. The design of the immunogenicity study M-000 is presented in Table 1.

TABLE 1
Immunogenicity Study M-000: Codon Optimized nCoV-S-RBD-Foldon

| Group | Antigens | SLA-LSQ | GPI-0100 | Alhydrogel | # mice |
|---|---|---|---|---|---|
| 1 | 5 μg nCoV-RBD-F-2 | 5 μg/2 μg | — | — | 10 |
| 2 | 5 μg nCoV-RBD-F-2 | — | 100 μg | — | 10 |
| 3 | 5 μg nCoV-RBD-F-2 | — | — | 120 μg | 10 |

Five mice were sacrificed and bled two weeks after dose two and 5 mice were sacrificed and bled two weeks after dose three. Serum was then assessed for nCoV-RBD antibody titers by ELISA and for virus nAbs using a MN assay with live SARS-CoV-2. Additionally, serum was tested in an RBD/ACE2 blocking assay.

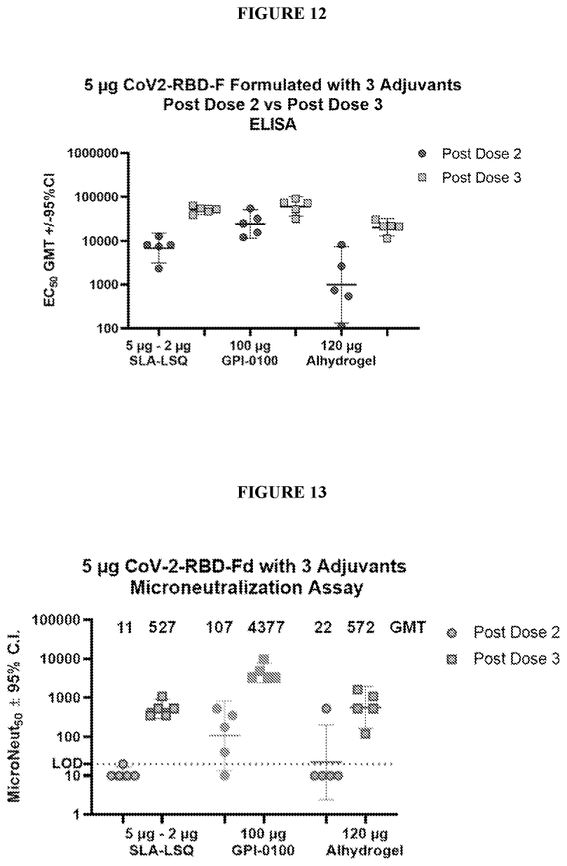

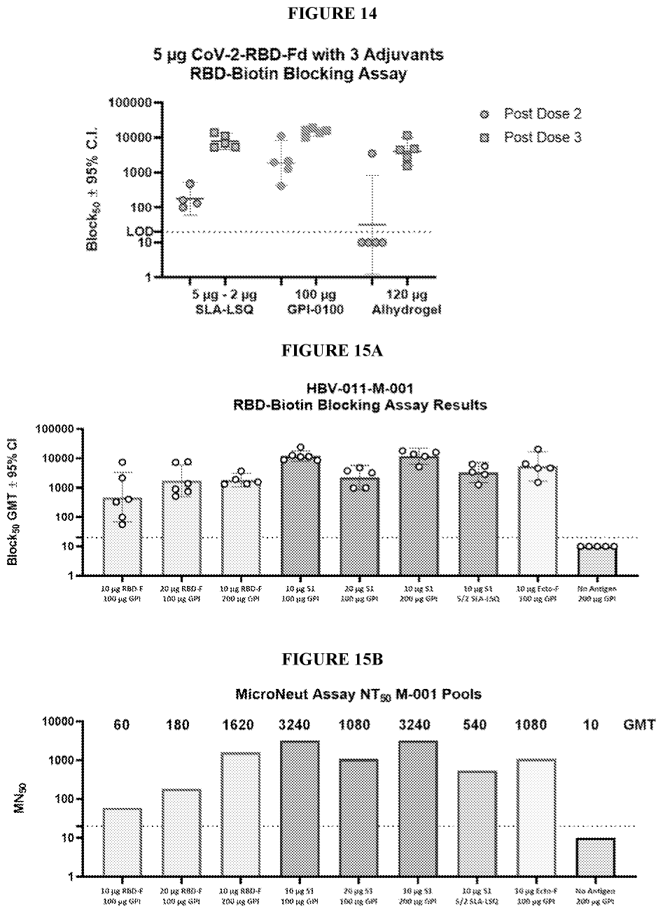

The ELISA results for the serum collected following two or three doses of nCoV-RBD-Foldon formulated with the 3 adjuvants are presented in FIG. 12. Serum from individual mice was diluted and were added to a plate coated with nCoV-RBD-Foldon. Bound antibody was detected with goat anti-mouse-AP conjugate. After the addition of substrate, color development was read after 1 hour. The results of the ELISA indicate that the nCoV-RBD-Foldon is immunogenic and the responses are most robust in the GPI-0100 group. The results of the MN assay of the post-dose 2 serum sample is presented in FIG. 13. Based on the 50% neutralization titers (NT— reported as GMT) for each group, the GPI-0100 formulated nCoV-RBD-Foldon results in the strongest response after 2 doses. To further assess the functionally of the serum samples from the nCoV-RBD-Foldon immunized mice, post-dose 2 and post-3 samples were tested for the ability to block the binding of RBD to the hACE2 receptor protein. In this assay, the hACE2 receptor protein, which is His-tagged, is captured on an ELISA plate with anti-His mAb. Dilutions of serum are mixed with a fixed amount of biotinylated nCoV-RBD-Foldon protein, after an incubation period the mixture is added to the plate with the hACE2 protein. Any nCoV-RBD-Foldon that binds to the hACE2 protein is detected with a streptavidin-alkaline phosphatase conjugate. The results of the blocking assay are presented in FIG. 14. The serum from the immunized mice are able to block the binding of biotinylated nCoV-RBD-Foldon protein to varying degrees, with the GPI-0100 group have the greatest blocking capacity. These results correlate with the PRNT results and confirm that the serum produced following immunization with nCoV-RBD-Foldon is able to block the RBD/ACE2 interaction.

### Example 7

### Immunogenic Evaluation of Codon Optimized SARS-Cov-2 Spike RBD, RBD-Foldon, S1, and Ecto-Foldon Proteins in Mice

The immunogenicity of the Drosophila S2 expressed codon optimized nCoV spike protein subunits, RBD, RBD-Foldon, S1, and Ecto-Foldon, were evaluated in Balb/c mice in 2 different studies. In addition to evaluation of the various nCoV spike protein subunits, varying amounts of proteins, and several different adjuvants were evaluated. Mice were immunized intra-muscularly with 2 doses separated by 3 weeks. A quantity of 10.0 μg or 20.0 μg nCoV spike protein subunits was used. The adjuvants tested were GPI-0100, SLA-LSQ, SLA-SE, QS21, CpG, and QS21+CpG. The design of the immunogenicity study M-001 and M-002 are presented in Table 2 and Table 3.

TABLE 2
Immunogenicity Study M-001: Codon Optimized
nCoV RBD-Foldon, S1, Ecto-Foldon

| Group | Antigen | GPI-0100 | SLA-LSQ | # mice |
|---|---|---|---|---|
| 1 | 10 μg nCoV-RBD-F-2 | 100 μg | — | 6 |
| 2 | 20 μg nCoV-RBD-F-2 | 100 μg | — | 6 |
| 3 | 10 μg nCoV-RBD-F-2 | 200 μg | — | 6 |
| 4 | 10 μg nCoV-S1-6 | 100 μg | — | 6 |
| 5 | 12 μg nCoV-S1-6 | 100 μg | — | 5 |
| 6 | 10 μg nCoV-S1-6 | 200 μg | — | 5 |
| 7 | 10 μg nCoV-S1-6 | — | 5 μg/2 μg | 5 |
| 8 | 10 μg nCoV-Ecto-F-6 | 100 μg | — | 5 |
| 9 | none | 200 μg | — | 5 |

TABLE 3
Immunogenicity Study M-002: Codon Optimized nCoV
RBD, RBD-Foldon, S1, and Ecto-Foldon

| | | GPI- | QS- | | SLA- | # |
|---|---|---|---|---|---|---|
| Group | Antigens | 0100 | 21 | CpG | SE | mice |
| 1 | 10 μg nCoV-RBD | 100 μg | — | — | — | 6 |
| 2 | 10 μg nCoV-RBD-F-2 | 100 μg | — | — | — | 6 |
| 3 | 10 μg nCoV-S1-6 | 100 μg | — | — | — | 6 |
| 4 | 20 μg nCoV-RBD | 200 μg | — | — | — | 6 |
| 5 | 20 μg nCoV-S1-6 | 200 μg | — | — | — | 6 |
| 6 | 10 μg nCoV-S1-6 | — | 10 μg | — | — | 6 |
| 7 | 10 μg nCoV-S1-6 | — | 10 μg | 20 μg | — | 6 |
| 8 | 10 μg nCoV-RBD | 100 μg | — | — | — | 6 |
| | 10 μg nCoV-S1-6 | | | | | |
| 9 | 10 μg nCoV-S1-6 | 200 μg | — | — | — | 6 |
| | 10 μg nCoV-RBD | | | | | |
| 10 | 20 μg nCoV-S1-6 | — | — | — | 20 μg | 6 |
| 11 | none | — | 10 μg | 20 μg | — | 6 |

Mice were sacrificed and bled two weeks after the second dose. Serum was then assessed for nCoV-RBD antibody titers by ELISA and for virus nAbs using a MN assay with live SARS-CoV-2. Additionally, serum was tested in an RBD/ACE2 blocking assay.

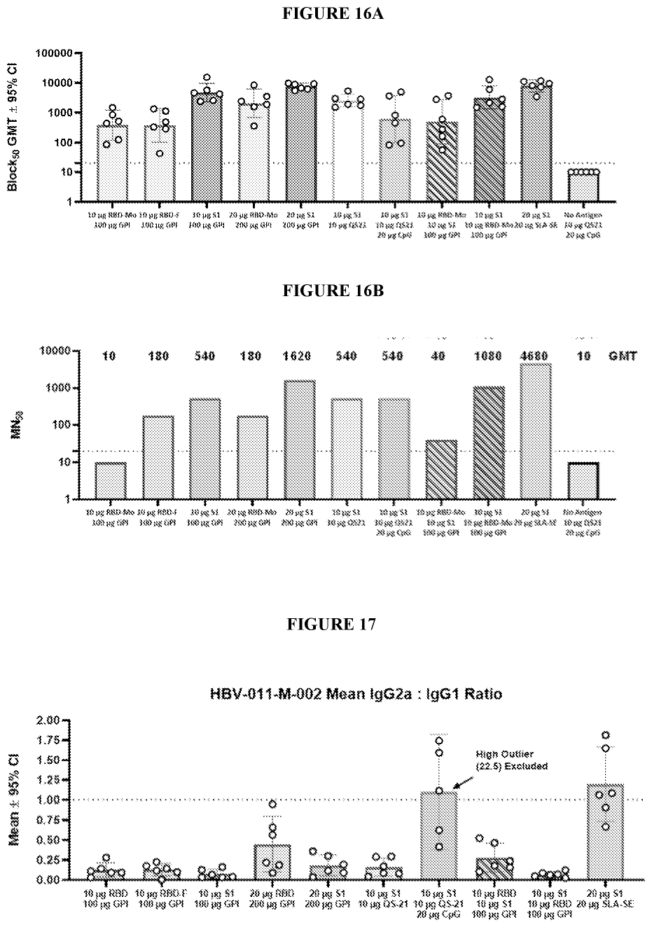

For the determination of total IgG, IgG1, and IgG2a by ELISA, serum from individual mice was diluted and added to a plate coated with nCoV-RBD-Foldon. Bound antibody was detected with goat anti-mouse-IgG-AP conjugates. After the addition of substrate, color development was read after 1 hour. For the determination of 50% micro-neutralization titers (MN₅₀), pooled serum for each group was tested in duplicate and the average titer is reported. For the determination of serum ability to block the binding of RBD to the hACE2 receptor protein, serum from individual mice was diluted and mixed with a fixed amount of biotinylated nCoV-RBD-Foldon protein and incubation for a set time, the mixture was then added to a plate with bound hACE2 protein. The amount of bound biotinylated nCoV-RBD-Foldon was detected with a streptavidin-alkaline phosphatase conjugate. The blocking ability of serum was compared to signal generated by the fixed amount of biotinylated nCoV-RBD-Foldon protein with no serum added. The GMT of the serum dilution for each group that blocks 50% of binding is reported. The results of the blocking assay and MN assay for the serum samples for study M-001 and M-002 are presented in FIG. 15A, FIG. 15B, FIG. 16A and FIG. 16B, respectively. This data indicates that in terms of immunogenic potential, specifically, the ability to elicit high levels of ACE2 blocking Abs and nAbs, the recombinant S protein subunits can be ranked from highest to lowest as follows: S1, Ecto, RBD. In terms of specific subunit/adjuvant combinations and immunogenic potential, the combination of S1/SLA-SE resulted in the strongest immune response.

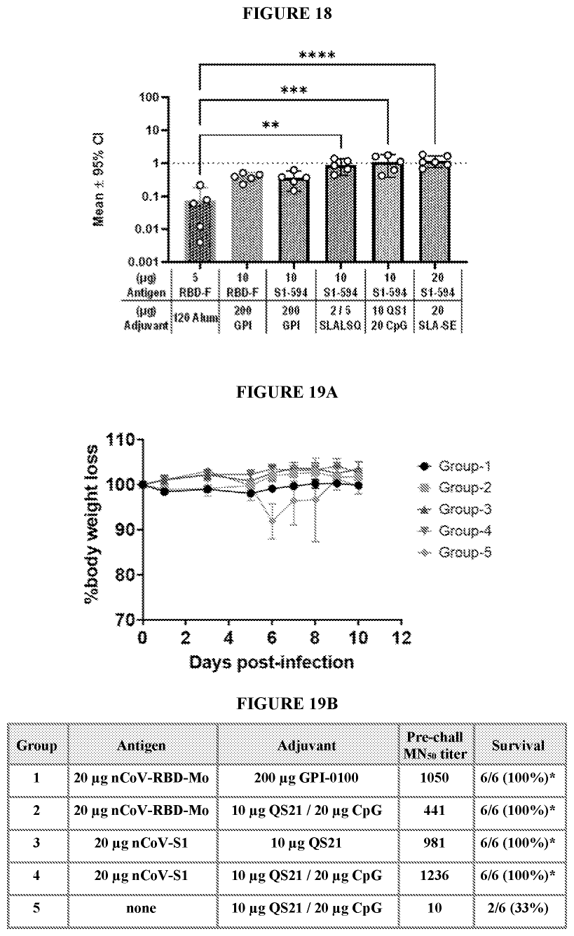

The ELISA IgG2a/IgG1 ratio results are reported in FIG. 17 for study M-002. These results indicate that only the S1 antigen with the SLA-SE adjuvant, or QS-21/CpG adjuvant combination results in a balanced response as indicated by a IgG2a/IgG1 ratio >1. The IgG2a/IgG1 ratios for select groups from studies M-000, M-001, M-002 are shown in FIG. 18. These results demonstrate the range of responses from Alum at the lowest and SLA-SE at the highest.

The evaluation of the various nCoV spike protein subunits at different amounts and with a variety of adjuvants all resulted in moderate to robust immune responses after 2 doses. In general, 20 μg of protein resulted in higher responses compared to a similar group with 10 μg of protein. For the various nCoV spike subunit proteins, the responses were highest for S1, followed by Ecto, followed by RBD. The combination of S1 and SLA-SE resulted in the strongest response (Study M-002, group 10). The MN results and the ACE2 blocking results are in good agreement. The combination of S1 and SLA-SE also resulted in the most balanced response as determined by the assessment of a IgG2a/IgG1 ratio.

### Example 8

### Protective Efficacy of Codon Optimized SARS-Cov-2 Spike RBD and S1 Proteins in hACE2 Transgenic Mice

The protective efficacy of Drosophila S2 expressed codon optimized nCoV spike protein subunits, RBD and S1 were evaluated in hACE2 transgenic mice (AC70) in 2 different virus challenge studies. In the first study (M-003), RBD and S1 proteins were evaluated. This study utilized 3 adjuvants, GPI-0100, QS21, and QS21+CpG. The M-003 study design is presented in Table 4. In the second study (M-005), the S1 protein was evaluated at 2 amounts, 10 and 20 μg. This study only utilized the adjuvant SLA-SE. The M-005 study design is presented in Table 5. Mice were immunized intra-muscularly with 2 doses separated by 3 weeks.

TABLE 4
Challenge Study M-003: Evaluation of protective efficacy
for codon optimized nCoV RBD and S1 with various adjuvants
in hACE2 transgenic mice, challenge dose 100 × LD₅₀.

| Group | Antigens | GPI-0100 | QS-21 | CpG | # mice |
|---|---|---|---|---|---|
| 1 | 10 μg nCoV-RBD | 200 μg | — | — | 6 |
| 2 | 10 μg nCoV-RBD | — | 10 μg | 20 μg | 6 |
| 3 | 10 μg nCoV-S1-6 | — | 10 μg | — | 6 |
| 4 | 10 μg nCoV-S1-6 | — | 10 μg | 20 μg | 6 |
| 5 | none | — | 10 μg | 20 μg | 6 |

TABLE 5
Challenge Study M-005: Evaluation of protective efficacy
for codon optimized nCoV S1 with SLA-SE adjuvant in hACE2 transgenic
mice, challenge dose 1000 × LD₅₀.

| Group | Antigens | SLA-SE | # mice |
|---|---|---|---|
| 1 | 10 μg nCoV-S1-6 | 20 μg | 6 |
| 2 | 20 μg nCoV-S1-6 | 20 μg | 6 |
| 3 | none | — | 6 |

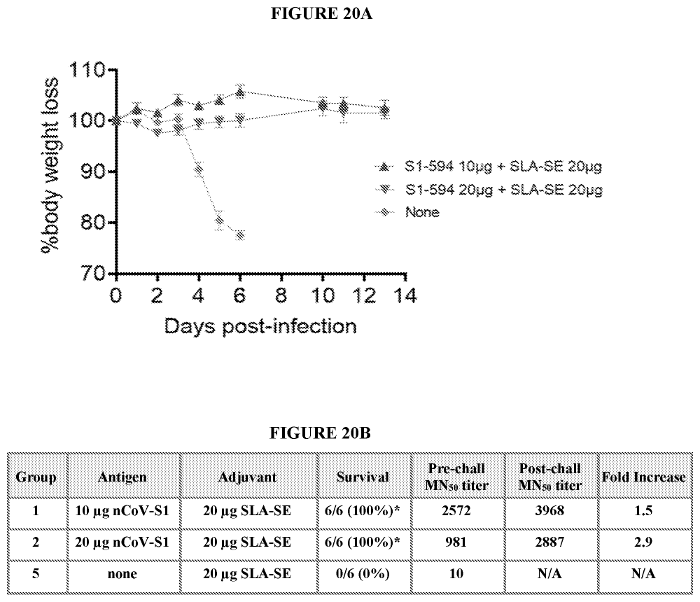

In both studies the mice were challenged 2 weeks after the second dose with SARS-CoV-2 wild type virus, USA WA1/2020 isolate. In challenge study M-003, mice were challenged with 31×LD₅₀ (100 TCID₅₀). In challenge study M-005, mice were challenged with 1000×LD₅₀ (3.2×10³ TCID₅₀). Following challenge, mice were monitored for signs of disease onset and body weights were recorded. If disease progressed to a defined state, or at a weight loss of greater than 20%, mice were euthanized. The results of percent body weight change and a summary table of results for MN₅₀ titers and percent survival for studies M-003 and M-005 are presented in FIG. 19A and FIG. 19B and FIG. 20A and FIG. 20B, respectively.

In study M-003, both the RBD and the 51 proteins resulted in appreciable MN₅₀ titers regardless of adjuvant used (441 to 1236). The RBD MN₅₀ titer with GPI-0100 is 2-fold greater than the titer with QS21+CpG, 1050 and 441, respectively. For 51 groups, there is not much difference in the MN₅₀ titer for QS21 alone or QS21+CpG, 981 and 1236, respectively. In terms of percent body weight change, the RBD groups had slight decreases, but recovered, while there was no weight loss in the 51 groups. All 4 groups had 100% survival, while the control group had 33% survival. While the protection in the immunized groups is significant relative to the control group, the lack of 100% mortality in the control group limits the interpretation of this study.

In study M-005, the two S1 protein groups resulted in appreciable MN₅₀ titers, with the 10 μg group having a titer greater than 2-fold that of the 20 μg group, 2572 and 981, respectively. These titers suggest that the antigen to adjuvant ratio impacts the titers. To ensure 100% mortality in the control group, the virus challenge was increased to 1000×LD₅₀, which was achieved. Both S1 groups were fully protected from this higher virus challenge. Importantly, the mice in the two S1 groups showed no signs of disease. In the 10 μg group there was no decrease in body weight, whereas in the 20 μg group, there was a slight decrease in body weight though day 2 post-challenge, but recovered after the initial decrease. Furthermore, the MN₅₀ titers post challenge did not increase substantially from the pre-challenge titers, 1.5 and 2.9 fold for the 10 μg and 20 μg groups, respectively. These limited increases in MN₅₀ titers post-challenge indicate that there was limited virus replication post challenge. Overall, study M-005 demonstrates that the combination of the nCoV spike S1 subunit protein in combination with the SLA-SE adjuvant provides solid protection against challenge with a lethal dose of SARS-CoV-2.

Although the invention has been described with reference to the above examples, it will be understood that modifications and variations are encompassed within the spirit and scope of the invention. Accordingly, the invention is limited only by the following claims.

1. An isolated nucleic acid sequence selected from the group consisting of SEQ ID NO: 1, 2, 3, 4, 5 and 6.

2. An isolated amino acid sequence encoded by a nucleic acid sequence of claim 1.

3. The isolated amino acid sequence of claim 2, wherein the sequence comprises SEQ ID NO: 9, 10, 11, 12, 13 or 14.

4. An expression vector comprising a nucleic acid sequence encoding a SARS-CoV-2 spike (S) protein, wherein the nucleic acid sequence comprises SEQ ID NO: 1, 2, 3, 4, 5 or 6.

5. The vector of claim 4, wherein the vector is a Drosophila melanogaster expression vector.

6. The vector of claim 4, wherein the vector has a nucleic acid sequence comprising SEQ ID NO: 7.

7. The vector of claim 4, wherein the SARS-CoV-2 S protein has an amino acid sequence as set forth in SEQ ID NO: 9, 10, 11, 12, 13 or 14.

8. A method of producing a protein in vitro comprising Drosophila melanogaster cells transformed with the vector of claim 4 and culturing the cells under conditions to produce the protein.

9. The method of claim 8, wherein the Drosophila melanogaster cells are Schneider 2 (S2) cells.

10. A vaccine composition comprising:
(a) an effective amount of a SARS-CoV-2 spike (S) protein, wherein the S protein is encoded by a nucleic acid sequence as set forth in SEQ ID NO: 1, 2, 3, 4, 5 or 6, and
(b) an effective amount of an adjuvant.

11. The vaccine of claim 10, wherein the S protein is the 51 subunit protein.

12. The vaccine of claim 11, wherein the 51 subunit protein is encoded by a nucleic acid sequence as set forth in SEQ ID NO: 3.

13. The vaccine of claim 10, wherein the adjuvant is selected from the group consisting of GPI-0100, synthetic lipid A (SLA) in a stable oil-in water emulsion (SE) (SLA-SE), QS21, QS21 combined with SLA to form a liposome formulation (SLA-LSQ), and QS21+CpG.

14. The vaccine of claim 11, wherein the adjuvant is SLA-SE.

15. The vaccine of claim 10, wherein the S protein is a 51 subunit protein encoded by a nucleic acid sequence as set forth in SEQ ID NO: 3, and the adjuvant is SLA-SE.

16. The vaccine of claim 10, wherein the S protein is recombinantly produced and expressed in insect host cells.

17. The vaccine of claim 10, wherein the S protein is recombinantly produced and expressed in Drosophila melanogaster Schneider 2 (S2) cells.

18. The vaccine of claim 10, further comprising a pharmaceutically acceptable excipient or carrier.

19. A method of preventing a SARS-CoV-2 entry into a cell comprising contacting the cell with an effective amount of the vaccine of claim 10, thereby preventing SARS-CoV-2 entry into the cell.

20. A method of stimulating a protective immune response in a subject comprising administering to the subject an effective amount of the vaccine of claim 10, thereby stimulating a protective immune response.

21. The method of claim 17, wherein the immune response is a balanced immune response.

22. The method of claim 18, wherein the balanced immune response is characterized by a IgG2a:IgG1 ratio that is equal or greater than 1.

23-37. (canceled)


SEQUENCE LISTING (18 sequences)

SEQ ID NO: 1 - protein, 1134 aa, Artificial Sequence
  Description of Artificial Sequence Synthetic polypeptide
QCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDS

SEQ ID NO: 2 - protein, 1134 aa, Artificial Sequence; identical to SEQ ID NO: 1
  Description of Artificial Sequence Synthetic polypeptide

SEQ ID NO: 3 - protein, 581 aa, Artificial Sequence
  Description of Artificial Sequence Synthetic polypeptide
QCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGG

SEQ ID NO: 4 - protein, 277 aa, Artificial Sequence
  Description of Artificial Sequence Synthetic polypeptide
FRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGG

SEQ ID NO: 5 - protein, 277 aa, Artificial Sequence; identical to SEQ ID NO: 4
  Description of Artificial Sequence Synthetic polypeptide

SEQ ID NO: 6 - protein, 292 aa, Artificial Sequence
  Description of Artificial Sequence Synthetic polypeptide
QCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKS

SEQ ID NO: 7 - DNA, 725 bp, Artificial Sequence
  Description of Artificial Sequence Synthetic polynucleotide
ggtaccgttgcaggacaggatgtggtgcccgatgtgactagctctttgctgcaggccgtcctatcctctggttccgataagagacccagaactccggccccccaccgcccaccgccacccccatacatatgtggtacgcaagtaagagtgcctgcgcatgccccatgtgccccaccaagagttttgcatcccatacaagtccccaaagtggagaaccgaaccaattcttcgcgggcagaacaaaagcttctgcacacgtctccactcgaatttggagccggccggcgtgtgcaaaagaggtgaatcgaacgaaagacccgtgtgtaaagccgcgtttccaaaatgtataaaaccgagagcatctggccaatgtgcatcagttgtggtcagcagcaaaatcaagtgaatcatctcagtgcaactaaagggggaatctagaaacaacatgaagaccattatcgccctgtcgtacatcttttgcctggtgttcgctagctctagctagaggctcgaggcccttcgaaggatccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaag

SEQ ID NO: 8 - protein, 1273 aa, Artificial Sequence
  Description of Artificial Sequence Synthetic polypeptide
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 9 - protein, 1137 aa, Artificial Sequence
  Description of Artificial Sequence Synthetic polypeptide
SSDQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPGSARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDS

SEQ ID NO: 10 - protein, 584 aa, Artificial Sequence
  Description of Artificial Sequence Synthetic polypeptide
SSDQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGG

SEQ ID NO: 11 - protein, 280 aa, Artificial Sequence
  Description of Artificial Sequence Synthetic polypeptide
SSDFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGG

SEQ ID NO: 12 - protein, 1172 aa, Artificial Sequence
  Description of Artificial Sequence Synthetic polypeptide
SSDQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPGSARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSGGSGGPGSGYIPEAPRDGQAYVRKDGEWVLLSTFL

SEQ ID NO: 13 - protein, 315 aa, Artificial Sequence
  Description of Artificial Sequence Synthetic polypeptide
SSDFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGGGSGGPGSGYIPEAPRDGQAYVRKDGEWVLLSTFL

SEQ ID NO: 14 - protein, 295 aa, Artificial Sequence
  Description of Artificial Sequence Synthetic polypeptide
SSDQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKS

SEQ ID NO: 15 - DNA, 3822 bp, Artificial Sequence
  Description of Artificial Sequence Synthetic polynucleotide
atgtttgtttttcttgttttattgccactagtctctagtcagtgtgttaatcttacaaccagaactcaattaccccctgcatacactaattctttcacacgtggtgtttattaccctgacaaagttttcagatcctcagttttacattcaactcaggacttgttcttacctttcttttccaatgttacttggttccatgctatacatgtctctgggaccaatggtactaagaggtttgataaccctgtcctaccatttaatgatggtgtttattttgcttccactgagaagtctaacataataagaggctggatttttggtactactttagattcgaagacccagtccctacttattgttaataacgctactaatgttgttattaaagtctgtgaatttcaattttgtaatgatccatttttgggtgtttattaccacaaaaacaacaaaagttggatggaaagtgagttcagagtttattctagtgcgaataattgcacttttgaatatgtctctcagccttttcttatggaccttgaaggaaaacagggtaatttcaaaaatcttagggaatttgtgtttaagaatattgatggttattttaaaatatattctaagcacacgcctattaatttagtgcgtgatctccctcagggtttttcggctttagaaccattggtagatttgccaataggtattaacatcactaggtttcaaactttacttgctttacatagaagttatttgactcctggtgattcttcttcaggttggacagctggtgctgcagcttattatgtgggttatcttcaacctaggacttttctattaaaatataatgaaaatggaaccattacagatgctgtagactgtgcacttgaccctctctcagaaacaaagtgtacgttgaaatccttcactgtagaaaaaggaatctatcaaacttctaactttagagtccaaccaacagaatctattgttagatttcctaatattacaaacttgtgcccttttggtgaagtttttaacgccaccagatttgcatctgtttatgcttggaacaggaagagaatcagcaactgtgttgctgattattctgtcctatataattccgcatcattttccacttttaagtgttatggagtgtctcctactaaattaaatgatctctgctttactaatgtctatgcagattcatttgtaattagaggtgatgaagtcagacaaatcgctccagggcaaactggaaagattgctgattataattataaattaccagatgattttacaggctgcgttatagcttggaattctaacaatcttgattctaaggttggtggtaattataattacctgtatagattgtttaggaagtctaatctcaaaccttttgagagagatatttcaactgaaatctatcaggccggtagcacaccttgtaatggtgttgaaggttttaattgttactttcctttacaatcatatggtttccaacccactaatggtgttggttaccaaccatacagagtagtagtactttcttttgaacttctacatgcaccagcaactgtttgtggacctaaaaagtctactaatttggttaaaaacaaatgtgtcaatttcaacttcaatggtttaacaggcacaggtgttcttactgagtctaacaaaaagtttctgcctttccaacaatttggcagagacattgctgacactactgatgctgtccgtgatccacagacacttgagattcttgacattacaccatgttcttttggtggtgtcagtgttataacaccaggaacaaatacttctaaccaggttgctgttctttatcaggatgttaactgcacagaagtccctgttgctattcatgcagatcaacttactcctacttggcgtgtttattctacaggttctaatgtttttcaaacacgtgcaggctgtttaataggggctgaacatgtcaacaactcatatgagtgtgacatacccattggtgcaggtatatgcgctagttatcagactcagactaattctcctcggcgggcacgtagtgtagctagtcaatccatcattgcctacactatgtcacttggtgcagaaaattcagttgcttactctaataactctattgccatacccacaaattttactattagtgttaccacagaaattctaccagtgtctatgaccaagacatcagtagattgtacaatgtacatttgtggtgattcaactgaatgcagcaatcttttgttgcaatatggcagtttttgtacacaattaaaccgtgctttaactggaatagctgttgaacaagacaaaaacacccaagaagtttttgcacaagtcaaacaaatttacaaaacaccaccaattaaagattttggtggttttaatttttcacaaatattaccagatccatcaaaaccaagcaagaggtcatttattgaagatctacttttcaacaaagtgacacttgcagatgctggcttcatcaaacaatatggtgattgccttggtgatattgctgctagagacctcatttgtgcacaaaagtttaacggccttactgttttgccacctttgctcacagatgaaatgattgctcaatacacttctgcactgttagcgggtacaatcacttctggttggacctttggtgcaggtgctgcattacaaataccatttgctatgcaaatggcttataggtttaatggtattggagttacacagaatgttctctatgagaaccaaaaattgattgccaaccaatttaatagtgctattggcaaaattcaagactcactttcttccacagcaagtgcacttggaaaacttcaagatgtggtcaaccaaaatgcacaagctttaaacacgcttgttaaacaacttagctccaattttggtgcaatttcaagtgttttaaatgatatcctttcacgtcttgacaaagttgaggctgaagtgcaaattgataggttgatcacaggcagacttcaaagtttgcagacatatgtgactcaacaattaattagagctgcagaaatcagagcttctgctaatcttgctgctactaaaatgtcagagtgtgtacttggacaatcaaaaagagttgatttttgtggaaagggctatcatcttatgtccttccctcagtcagcacctcatggtgtagtcttcttgcatgtgacttatgtccctgcacaagaaaagaacttcacaactgctcctgccatttgtcatgatggaaaagcacactttcctcgtgaaggtgtctttgtttcaaatggcacacactggtttgtaacacaaaggaatttttatgaaccacaaatcattactacagacaacacatttgtgtctggtaactgtgatgttgtaataggaattgtcaacaacacagtttatgatcctttgcaacctgaattagactcattcaaggaggagttagataaatattttaagaatcatacatcaccagatgttgatttaggtgacatctctggcattaatgcttcagttgtaaacattcaaaaagaaattgaccgcctcaatgaggttgccaagaatttaaatgaatctctcatcgatctccaagaacttggaaagtatgagcagtatataaaatggccatggtacatttggctaggttttatagctggcttgattgccatagtaatggtgacaattatgctttgctgtatgaccagttgctgtagttgtctcaagggctgttgttcttgtggatcctgctgcaaatttgatgaagacgactctgagccagtgctcaaaggagtcaaattacattacacataa

SEQ ID NO: 16 - protein, 4 aa, Artificial Sequence
  Description of Artificial Sequence Synthetic peptide
RRAR

SEQ ID NO: 17 - protein, 4 aa, Artificial Sequence
  Description of Artificial Sequence Synthetic peptide
GSAR

SEQ ID NO: 18 - protein, 4 aa, Artificial Sequence
  Description of Artificial Sequence Synthetic peptide
SSDQ